(19) United States
(12) Patent Application Publication
(10) Pub. No.: US 2020/0359942 A1
(43) Pub. Date: Nov. 19, 2020

(54) LASER-ENABLED LAB ON SKIN

(71) Applicant: California Institute of Technology, Pasadena, CA (US)

(72) Inventors: Wei Gao, Pasadena, CA (US); Yiran Yang, Pasadena, CA (US)

(21) Appl. No.: 16/875,834

(22) Filed: May 15, 2020

Related U.S. Application Data
(60) Provisional application No. 62/848,676, filed on May 16, 2019.

(51) Int. Cl. A61B 5/1477; A61B 5/053; A61B 5/20; A61B 5/00

(52) U.S. Cl. CPC A61B 5/1477; A61B 5/0537; A61B 5/207; B23K 26/362; A61B 5/4277; A61B 2562/125; A61B 5/4266

(57) ABSTRACT

A lab-on-skin biosensor for detecting target molecule and vital sign monitoring, a method of manufacturing, and a method of using the same, wherein the lab-on-skin biosensor is fabricated with a microfluidics layer, a moisture resistant layer, a multimodal sensing layer comprising an electrode, and a logic circuit that may include a processor and non-transitory memory with computer executable instructions embedded thereon.

### CROSS-REFERENCE TO RELATED APPLICATIONS

This application claims priority to U.S. provisional application No. 62/848,676 titled “Laser-Enabled Lab On Skin” and filed May 16, 2019, the contents of which are incorporated herein by reference in its entirety.

### BACKGROUND

Circulating molecules including nutrients, drugs, hormones, and metabolites, are associated with various health conditions and are useful for the diagnosis, treatment, and monitoring of personal health. Wearable biosensors that can monitor the concentration of various target molecules in a user are necessary to realize personalized health management.

Medical biosensors have traditionally required invasive blood sampling for effective capture of concentrations of a target molecule in a user. Within the past few years, non-invasive portable biosensors have appeared that offer competitive metabolite concentration analysis through sampling sweat, tears, and saliva, and/or offer monitoring of vital signs including heartrate and body temperature. However, such devices require complicated and expensive fabrication to provide accurate metabolite concentration analysis, and are often too large to provide continuous comfortable wear for the user. Accordingly, there is a need for cost-effective, mass-producible wearable biosensor that is non-invasive but allows for continuous, and accurate real-time physiological data collection of changes in a user's health status with molecular data and vital signs.

### SUMMARY

The technology disclosed herein is directed to target molecule analysis and multiplexed vital sign monitoring. The wearable lab-on-skin biosensor and methods disclosed herein enable the cost-effective and continuous real-time data collection of a target molecule in a biological sample and offer monitoring of the physiological responses and vital signs of a user. For example, the biological sample may include blood, sweat, saliva, urine, or other substance excreted from an organism. In some examples, the organism could be a human or an animal.

In an example embodiment of the disclosed technology, a lab-on-skin biosensor for detecting a target molecule in a biological sample includes a microfluidics layer, a moisture resistant layer, a multimodal sensing layer comprising an electrode, and a logic circuit with a processor and a non-transitory memory with computer executable instructions embedded thereon. In some examples, the microfluidics layer may comprise multiple microchannels transversely oriented to channel a biological sample from a first surface of the microfluidics layer to a second surface of the microfluidics layer. The moisture resistant layer may couple to the first surface of the microfluidics layer and may comprise an aperture to enable the biological sample to enter the microchannels of the microfluidics layer. In some embodiments, the moisture resistant layer may be polyethylene terephthalate (PET). In some examples, the multimodal sensing layer may be fluidically coupled to the second surface of the microfluidics layer to receive the biological sample from the microchannels. The electrode, for example, may be configured to detect a measurement of an electrical property corresponding to a target molecule being present in the biological sample. In embodiments, the logic circuit may be electrically coupled to the electrode and the computer executable instructions may cause the processor to identify the electrical property detected with the electrode when the target molecule is present in the biological sample. A target molecule, for example, may include a specific protein, peptide, vitamin, amino acid, hormone, antibody, or drug metabolite.

In some embodiments, the multimodal sensing layer comprises a polymer, for example, polyimide film. In some embodiments, the multimodal sensing layer may be regenerated in-situ. In some embodiments, the electrode may also include a uniform redox probe, wherein the uniform redox probe is deposited on a surface of the electrode. In some embodiments, the electrode comprises a catalytically active substrate. In some embodiments, the catalytically active substrate is graphene. In embodiments, the electrical property may be an electrical current, an electrical voltage, or an electrical impedance.

In some embodiments, the computer executable instructions may include causing the processor to generate an indication identifying the presence of the target molecule based on the electrical property detected with the electrode. In some embodiments, the lab-on-skin biosensor may also include a display, wherein the computer executable instructions may cause the processor to output the indication identifying the presence of the target molecule to the display.

In an example of the embodiments, a method for manufacturing a lab-on-skin biosensor is disclosed. The lab-on-skin biosensor, for example, may include a microfluidics layer, a multimodal sensing layer comprising an electrode configured to detect a measurement of an electrical property corresponding to a target molecule being present in a biological sample, a moisture resistant layer, and a logic circuit. The method may include shaping the microfluidics layer to receive a biological sample, and laser scribing the electrode on a surface of the multimodal sensing layer.

In some embodiments, shaping the microfluidics layer to channel a biological sample comprises laser engraving multiple microchannels. In some embodiments, the method may include electrodepositing a conductive substance onto the surface of the electrode. In some embodiments, the method may include laser engraving an aperture onto a surface of the moisture resistant layer. In some embodiments, the multimodal sensing layer comprises polyimide film. In some embodiments, the electrode comprises graphene.

In an example of the embodiments, a method for detecting a target molecule in a biological sample using a lab-on-skin biosensor is disclosed. The lab-on-skin biosensor, for example, may include a microfluidics that may comprise multiple microchannels transversely oriented to channel a biological sample from a first surface of the microfluidics layer to a second surface of the microfluidics layer, a moisture resistant layer, a multimodal sensing layer comprising an electrode, and a logic circuit. In some embodiments, the method may include receiving, on a first surface of the microfluidics layer, a biological sample comprising the target molecule, such that the biological sample can be channeled from a first surface of the microfluidics layer to a second surface of the microfluidics layer; obtaining, with the electrode, a measurement of an electrical property of the target molecule; and generating, with the logic circuit, an indication that the target molecule is present in the biological sample based on the measurement of the electrical property.

In some embodiments, the method may also include sweeping the electrode to regenerate the multimodal sensing layer in-situ. In some embodiments, the method may also include depositing a uniform redox probe on a surface of the electrode.

In some embodiments, the electrical property may include an electrical current, an electrical property, and/or an electrical impedance.

In some embodiments, the biological sample may include, for example, sweat, urine, blood, or saliva. In some embodiments, the target molecule is, for example, a monoclonal antibody against an epitope of SARS-CoV-2.

In some embodiments, the target molecule may include an electroactive molecule. For example, the electroactive target molecule may include tryptophan, tyrosine, phenylalanine, dopamine, vitamin C, vitamin B6, vitamin B12, uric acid, mycophenolic acid, caffeine, methionine, cortisol, noradrenaline, or adrenaline.

In some embodiments, the target molecule further may include a non-electroactive molecule. For example, the non-electroactive target molecule may include leucine, iso-leucine, valine, busulfan, cyclophosphamide, creatinine, or urea.

Other features and aspects of the disclosure will become apparent from the following detailed description, taken in conjunction with the accompanying drawings, which illustrate, by way of example, the features in accordance with various embodiments. The summary is not intended to limit the scope of the invention, which is defined solely by the claims attached hereto.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a block diagram illustrating some components of a lab-on-skin biosensor, in accordance with various embodiments of the disclosure.

FIG. 2 illustrates by way of example, microfluidic sampling in accordance with various embodiments disclosed herein.

FIG. 3 illustrates, by way of example, several uses for the lab-on-skin biosensor in accordance with various embodiments of the disclosure.

FIG. 4 illustrates, by way of example, target molecule detection and quantification, in accordance with various embodiments of the disclosure.

FIG. 5 illustrates various uses of the lab-on-skin biosensor in accordance with various embodiments of the disclosure.

FIG. 6 illustrates various uses of the lab-on-skin biosensor in accordance with various embodiments of the disclosure.

FIG. 7 illustrates, by way of example, target molecule detection and quantification, in accordance with various embodiments of the disclosure.

FIG. 8 illustrates, by way of example, implementations of a lab-on-skin biosensor in accordance with various embodiments of the disclosure.

FIG. 9 illustrates circuitry that can be utilized in implementing architectures and methods, in accordance with various implementations of the disclosure.

FIG. 10 is an operational flow diagram illustrating an example method for manufacturing a biosensor.

FIG. 11 is an operational flow diagram illustrating an example method for using lab-on-skin biosensor, in accordance with implementations of the disclosure.

FIG. 12 illustrates a computer component that can be utilized in implementing architectures and methods, in accordance with various implementations of the disclosure.

FIG. 13 illustrates, by way of example, electrode optimization and characterization in accordance with various embodiments of the disclosure.

FIG. 14 illustrates, by way of example, electrode optimization and characterization in accordance with various embodiments of the disclosure.

FIG. 15 illustrates, by way of example, target molecule detection and quantification in accordance with various embodiments of the disclosure.

FIG. 16 illustrates, by way of example, target molecule detection and quantification in accordance with various embodiments of the disclosure.

FIG. 17 illustrates, by way of example, electrode optimization and characterization in accordance with various embodiments of the disclosure.

FIG. 18 illustrates, by way of example, electrode optimization and characterization in accordance with various embodiments of the disclosure.

FIG. 18 illustrates, by way of example, electrode optimization and characterization in accordance with various embodiments of the disclosure.

FIG. 19 illustrates, by way of example, electrode characterization in accordance with various embodiments of the disclosure.

FIG. 20 illustrates, by way of example, microfluidic sampling and target molecule detection and quantification, in accordance with various embodiments of the disclosure.

FIG. 21 illustrates, by way of example, electrode characterization and data processing in accordance with various embodiments of the disclosure.

FIG. 22 illustrates, by way of example, lab-on-skin biosensor validation, in accordance with various embodiments of the disclosure.

FIG. 23 illustrates, by way of example, lab-on-skin biosensor validation, in accordance with various embodiments of the disclosure.

FIG. 24 illustrates, by way of example, lab-on-skin biosensor validation, in accordance with various embodiments of the disclosure.

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION

Wearable biosensors offer tremendous potential for biomedical applications. Recent advancements in sensor technology has allowed wearable biosensors to become non-invasive, while still offering the same degree of accurate molecule analysis as their invasive predecessors. Obstacles for the new generation of wearable biosensors remain however, as they require expensive fabrication facilities to manufacture and technical skill to operate. Moreover, because of their present size and construction, they often fail to monitor physiological changes in a user's molecule concentrations and vital signs, while offering the user comfort and flexibility during continuous daily wear and use. To address the deficiencies that presently face the wearable biosensor field, the technology disclosed herein is directed to a low-cost, mass-producible, lab-on-skin platform that can perform molecule analysis and vital sign monitoring of the user.

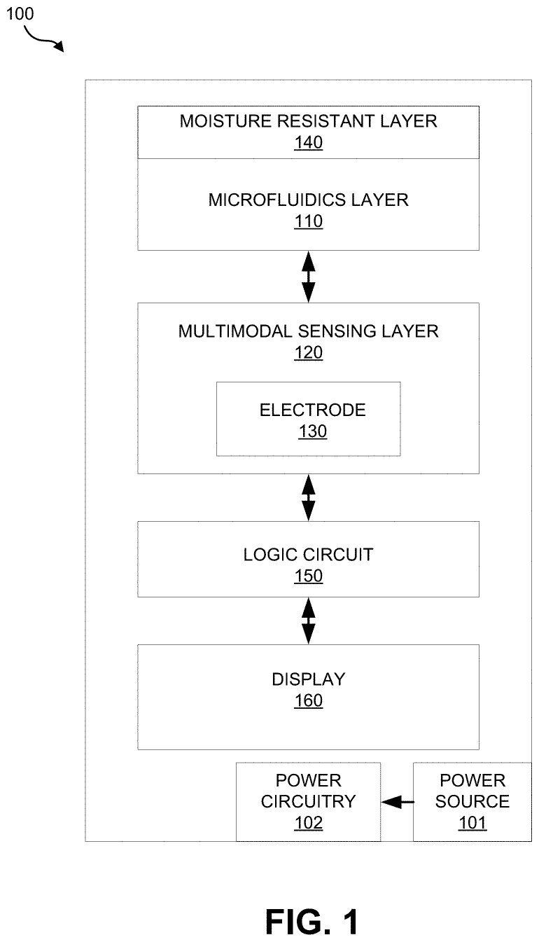

FIG. 1 is a block diagram illustrating some components of lab-on-skin biosensor 100, in accordance with various embodiments of the disclosure. The lab-on-skin biosensor 100 may include, for example, a microfluidics layer 110, a multimodal sensing layer 120 comprising an electrode 130, a logic circuit 140, and moisture resistant layer 150, and optionally, a display 160. The electrical components of lab-on-skin biosensor 100 may be powered by a power source 101 that connects to power circuitry 102 for distributing power. The power source 101 may be a battery, capacitor, or other power source known in the art. The power source 101 may be rechargeable (e.g., via a USB port and/or an AC/DC converter), and it should be appreciated that any suitable power source technologies may be used to power the components of lab-on-skin biosensor 100. For example, lithium-ion batteries, cell batteries, piezo or vibration energy harvesters, photovoltaic cells, AC/DC sources, or other like devices can be used.

During operation, lab-on-skin biosensor 100 may be introduced to a biological sample containing a target molecule. Microfluidics layer 110 is shaped to receive the biological sample from the user, and channel it to the multimodal sensing layer 120. Multimodal sensing layer 120 comprises an electrode 130 that detects the presence of a target molecule in the biological sample, such that logic circuit 140, which is electrically coupled to electrode 130, may generate an indication that the target molecule is present in the biological sample. In embodiments, lab-on-skin biosensor 100 may also include a moisture resistant layer 150 that is adhesively attached to microfluidics layer 110 and configured to funnel the biological sample through an inlet on its surface. In some embodiments, lab-on-skin biosensor 100 may also include a display 160 to display the indication.

In various embodiments, microfluidics layer 110 may be comprised of a material that may be shaped to have at least one microchannel or inlet through which a biological sample may flow. Such materials that may comprise microfluidics layer 110 include, for example, plastics (e.g., polyethylene film), ceramics, glass, metal, polymer, and/or wood (e.g., paper-based materials). In embodiments, microfluidics layer 110 may be adhesive such that it can attach to the user. In some embodiments, microfluidics layer 110 may be adhesive on both sides such that it can attach to the user and to other components of lab-on-skin biosensor 100 simultaneously. For example, in some embodiments, microfluidics layer 110 may be double-sided adhesive medical tape. In some embodiments, microfluidics layer 110 may comprise an adhesive elastomer (e.g., PDMS, Ecoflex).

In embodiments, manufacturing microfluidics layer 110 includes laser engraving a microchannel, reservoir, and/or inlet using a laser cutter, for example, a CO₂ laser. Laser engraving offers a great alternative for rapid and bulk manufacturing of the microfluidics layer 110. In some embodiments, microfluidics layer 110 is prepared using vector mode laser cutting. In some embodiments, raster mode laser cutting may be used. In some embodiments, at least one microchannel, reservoir, and/or inlet is laser-engraved into the surface of the microfluidics layer 110. In some embodiments, multiple microchannels, reservoirs, and/or inlets are engraved into the surface of microfluidics layer 110. In some embodiments, multiple microchannels, reservoirs, and/or inlets are laser engraved into and through microfluidics layer 110. In embodiments, laser engraving parameters for forming the microfluidics layer 110 may include: Power 1%, Speed 1.5%, PPI 1000 for reservoir outline and channels; and Power 2%, Speed 1%, PPI 1000 for inlet outlines, both in vector mode at focused height. It is to be understood that other laser engraving parameters may be used (e.g., power, speed, and/or PPI) depending on the application and material to be engraved.

The number of microchannels, reservoirs, and/or inlets of microfluidics layer 110 may vary in accordance with the use of the lab-on-skin biosensor. A person of ordinary skill in the art would appreciate how the flow rate of the biological sample may be impacted by the number of microchannels and/or inlets of microfluidics layer 110. Moreover, a person of ordinary skill would appreciate that the flow rate may also be impacted by the biological sample itself. In embodiments, between 1 to 10 microchannels and/or inlets may be engraved. In other embodiments, between 1-20 microchannels and/or inlets may be engraved. In still more embodiments, 1-100 microchannels and/or inlets may be engraved. The microchannels and/or inlets of microfluidics layer 110 may be transversely oriented to channel a biological sample from a first surface of microfluids layer 110 to a second surface of the microfluidics layer 110.

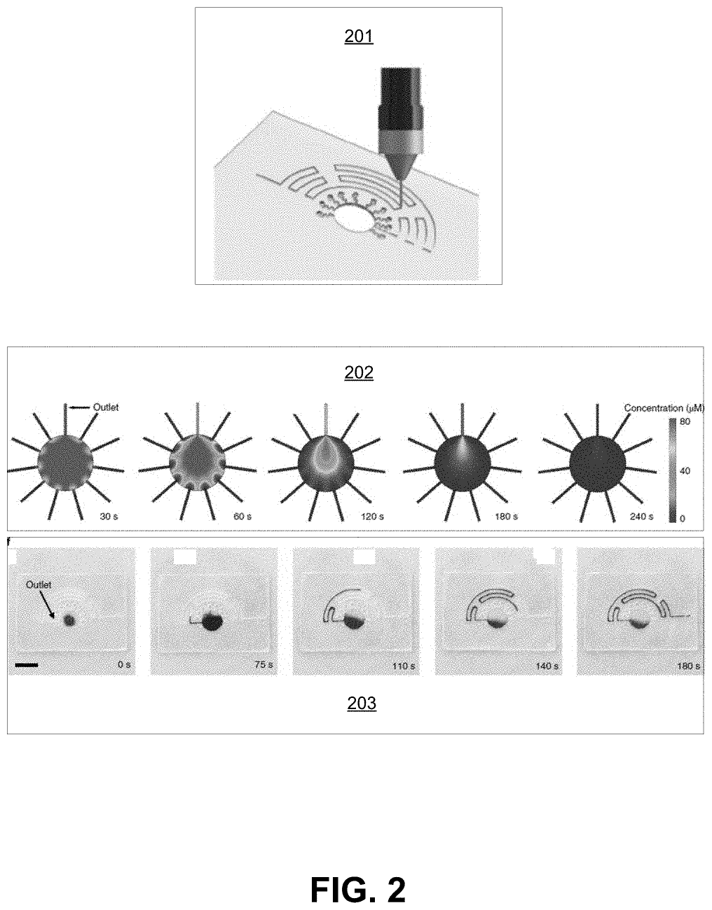

FIG. 2 illustrates by way of example, manufacturing and microfluidic sampling of microfluidics layer 110 in accordance with various embodiments disclosed herein. Frame 201, illustrates, by way of example, manufacturing of a microfluidics layer via vector-mode laser engraving. Frame 202, for example, provides simulated flow rate analysis of the average concentration of target molecule present in a biological sample using various total inlet flow rates over time (30-240 seconds). Microfluidic sweat sampling over time (0-170 seconds) during iontophoresis-induced sweat secretion can be seen in frame 203, for example. Microfluidics layer 110 may comprise double-sided medical adhesive tape. In some embodiments, microfluidics layer 110 may comprise an adhesive elastomer, including for example, PDMS and/or Ecoflex.

Referring again to FIG. 1, biosensor 100 may also include multimodal sensing layer 120. Multimodal sensing layer 120 may be fluidically coupled to a surface of microfluidics layer 110 in order to receive a biological sample. In embodiments, multimodal sensing layer 120 is fluidically coupled to the second surface of the microfluidics layer 110 to receive a biological sample from the microchannels, inlets, and/or reservoirs of microfluidics layer 110.

In embodiments, multimodal sensing layer 120 may comprise an electrode 130 that is configured to detect a measurement of an electrical property of a target molecule. In some embodiments, multimodal sensing layer 110 may comprise one electrode 130. In some embodiments, multimodal sensing layer 120 may comprise more than one electrode 130. In some embodiments, multimodal sensing layer 120 may comprise more than one electrode 130 allowing a user to monitor both chemical and physical properties (e.g., vital signs) of the biological sample and/or the user simultaneously or independently. In embodiments, multimodal sensing layer 120 may comprise a polymer, including, for example, polyimide (PI). Other materials may also be used to construct multimodal sensing layer 120 including metal.

In embodiments, electrode 130 may comprise a chemical sensor configured to detect a measurement of an electrical property of a target molecule. In some embodiments, electrode 130 may be a three-electrode chemical sensor designed to detect and measure various target molecules present in a biological sample. In some embodiments, electrode 130 may be a resistive sensor designed to detect various vital signs of the user. Vital signs that may be detected using electrode 130 include for example, body temperature, respiration rate, heartrate, pupil dilation, etc. In some embodiments, electrode 130 may be a resistive temperature sensor and/or a piezoresistive sensor.

In embodiments, electrode 130 may include a catalytically active substrate. Several types of electrode materials may be used in accordance with the embodiments disclosed herein. Each electrode material has its own advantages and disadvantages. Traditional electrode materials include, for example, graphite, platinum, gold, rhodium, indium, tin, copper, zinc, lead, and/or silver. More contemporary electrode materials include, for example, metallic nanowires, carbon nanotubes (CNTs), conductive polymers, and graphene (including graphene film). Graphene, for example, represents a promising conducting material and may be used as an electrode in a number of different applications including in transistors, light-emitting diodes, liquid crystal displays, molecular junction devices, touch screens, solar cells, and flexible devices. Graphene's advantages include its high charge mobility, transparency, mechanical strength, and flexibility. In embodiments, electrode 130 may include a catalytically active substrate, for example, graphene. In some embodiments, electrode 130 may include a graphite, platinum, gold, rhodium, indium, tin, copper, zinc, lead, and/or silver electrode. In some embodiments, other conductive materials may be used to form electrode 130, including for example, metallic nanowires, carbon nanotubes (CNTs), and/or conductive polymers. Such nanomaterials may also be used to increase the surface area and/or the signal response of the electrode.

In embodiments, electrode 130 may be laser scribed onto multimodal sensing layer 120. In some embodiments, multimodal sensing layer 120 may comprise polyimide (PI) substrate. In various implementations where multimodal sensing layer 120 comprises polyimide (PI), for example, laser scribing electrode 130 into the surface of multimodal sensing layer 120 results in electrode 130 being comprised of graphene.

In embodiments, electrode 130 may be manufactured using laser scribing (i.e., engraving and/or etching) technology. In some embodiments, electrode 130 may be laser scribed through vector mode and/or through raster mode. In implementations where electrode 130 may comprise a chemical sensor, for example, laser engraving using raster mode is preferred. In implementations where electrode 130 may comprise a resistive sensor (i.e., to detect heartrate, and other vital signs), laser engraving using vector mode is preferred. It is to be understood that multimodal sensing layer 120 may comprise more than one electrode 130, where one may be a chemical sensor and the other(s) may be a resistive (temperature based, and piezoresistive or “strain” based) sensor.

As depicted in FIG. 1, multimodal sensing layer 120 is fluidically coupled to microfluidics layer 110. In embodiments, multimodal sensing layer 120 comprises an electrode 130. Electrode 130 may be configured to detect a measurement of an electrical property of a target molecule in a biological sample that flows from the microfluidics layer 110. In embodiments, a measurement of an electrical property may become detectable by electrode 130 when the target molecule is present in the biological sample. In embodiments, electrode 130 may be configured to detect a measurement of an electrical property of a physical event (e.g., strain, pressure, resistance, and/or temperature).

In embodiments, the electrical property may include an electrical current. In some embodiments, the electrical property may include an electrical voltage. In some embodiments, the electrical property may include an electrical impedance. In embodiments, electrode 130 may be coupled to microfluidics layer 110 and may be configured to detect a measurement of an electrical property. In embodiments, a measurement of an electrical property may include reaching or meeting a threshold of an electrical property. In some embodiments, a measurement of an electrical property may include reaching or meeting a threshold on an electrical property such that when the threshold is reached or met, the measurement may be recorded and sent to the logic circuit for processing. In embodiments, a measurement may include a change in the electrical property. In some embodiments, a measurement may include a change in the electrical property such that when the change in electrical property may be detected, for example, from a baseline, the measurement may be sent to the logic circuit for processing. In some embodiments, a change in electrical property may include an increase or decrease in the electrical property from a certain baseline. In some embodiments, the change in the electrical current, electrical voltage, or electrical impedance, may include a change from a baseline level, or between two or more readings, depending on the assay performed.

In various embodiments disclosed herein, microfluidics layer 110 may be fluidically coupled to multimodal sensing layer 120, wherein multimodal sensing layer 120 comprises at least one electrode 130. In embodiments, multimodal sensing layer 120 comprises more than one electrode 130. In some embodiments, multimodal sensing layer 120 may be laser-engraved to produce electrode 130 on a surface of multimodal sensing layer 120.

In embodiments, electrode 130 may comprise graphene. Graphene serves as an excellent material for producing an electrode due to its unique electrochemical properties arising from the fast electron mobility, high current density, and ultra large surface area. In embodiments, electrode 130 may be manufactured from laser scribing the surface of multimodal sensing layer 120 in instances where multimodal sensing layer 120 comprises polyimide. In some embodiments, electrode 130 is a chemical sensor, including for example, a three-electrode chemical sensor. In some embodiments, electrode 130 is a chemical sensor that may detect a target molecule through differential pulse voltammetry (DPV) based on the amplitude of the oxidation current peak of the target molecule. In instances where electrode 130 is a three-electrode graphene sensor, electrode 130 can selectively catalyze the oxidation of various target molecules at specific potentials. In some embodiments, DPV peak amplitudes of various target molecules may be used to optimize laser engraving parameters when constructing electrode 130. In some embodiments, electrochemical impedance spectroscopy may also be used.

In some embodiments, electrode 130 may comprise a resistive sensor. In embodiments, electrode 130 is a resistive sensor comprising graphene. Indeed, due to its porous structure and high electron mobility, graphene serves as an excellent candidate for resistive sensors. For example, as temperature rises, the conductivity of the graphene increases due to its increased electron-phonon scattering and thermal velocity of electrons in the sandwiched layers. Moreover, when an external strain is applied, the porous structure of graphene allows the electrode to be compressed, resulting in decreased resistance. In some embodiments, electrode 130 may detect physical changes of the multimodal sensing layer 120, including for example, temperature differences, strain differences, and/or pressure differences.

In embodiments, electrode 130 may comprise a resistive sensor. In some embodiments wherein electrode 130 comprises a resistive sensor, the resistive sensor may comprise a resistive temperature sensor or a piezoresistive sensor. In some embodiments, electrode 130 may comprise a piezoresistive sensor that may detect external strain such as, for example, bending. In some embodiments, electrode 130 may comprise a resistive sensor manufactured from laser engraved graphene, wherein laser engraving comprises laser engraving in vector mode, a single continuous line.

Both material morphology and laser control play a role in manufacturing electrode 130 comprising a resistive sensor. For example, electrode 130 may comprise a piezoresistive sensor manufactured from laser engraving a fiber-like structure using a high dose of local laser power coupled with straight line engraving. The piezoresistive sensor of electrode 130 may be fabricated at a low laser speed for large strain response and high stability. In some embodiments, electrode 130 may comprise a resistive temperature sensor from laser engraving a compact structure using serpentine line design to minimize the strain interference on temperature measurement.

In various embodiments, multimodal sensing layer 120 may comprise more than one electrode 130. In some embodiments, multimodal sensing layer 120 may comprise for example, three electrodes 130, wherein the first electrode 130 comprises a laser engraved graphene-based chemical sensor; the second electrode 130 comprises a laser engraved graphene-based resistive temperature sensor; and wherein the third electrode 130 comprises a laser engraved graphene-based piezoresistive sensor.

In embodiments, electrode 130 may continuously sense a target molecule in the biological sample. For example, continuous sensing capabilities of electrode 130 may be achieved through continuous injection of a biological sample from the microfluidics layer 110. In embodiments, electrode 130 may continuously detect and measure the concentration of a target molecule through successive DPV scans over multiple cycle periods. In some embodiments, electrode 130 may continuously detect the body temperature of the user. In some embodiments, electrode 130 may continuously detect the heartrate and/or respiration rate of a user.

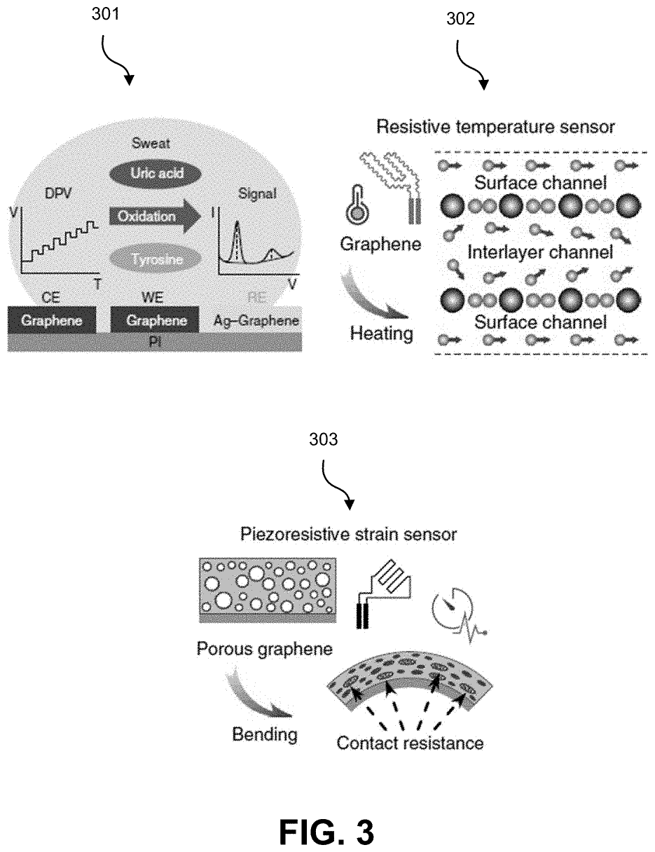

FIG. 3 illustrates, by way of example, various implementations and sensing capabilities of electrode 130. Chemical sensor 301 comprises a three-electrode based sensor (CE: counter electrode; WE: working electrode; and RE: reference electrode comprising silver/graphene) manufactured using raster mode laser engraving on polyimide (PI) film. Chemical sensor 301 may be used to detect target molecules in a biological sample, which here, by way of example, includes the continuous detection of uric acid and tyrosine in sweat using differential pulse voltammetry (DPV). In embodiments, detection of a target molecule using DPV may include use of other substrates including gold, platinum, carbon, or mercury. An example graphical representation, or an indication, of the quantity of target molecule detected is also depicted. Resistive temperature sensor 302 may detect and measure changes in the user's temperature over a period of time. Piezoresistive sensor 303 may detect bending and other physical pressures to determine vital signs, including heartrate and respiration rate of the user. In embodiments, upon detection by the electrode, the computer executable instructions embedded on the logic circuit cause the processor to identify the electrical property detected with the electrode. In some embodiments, the computer executable instructions further cause the processor to output the indication identifying the presence of the target molecule, and or physical measurement to the display.

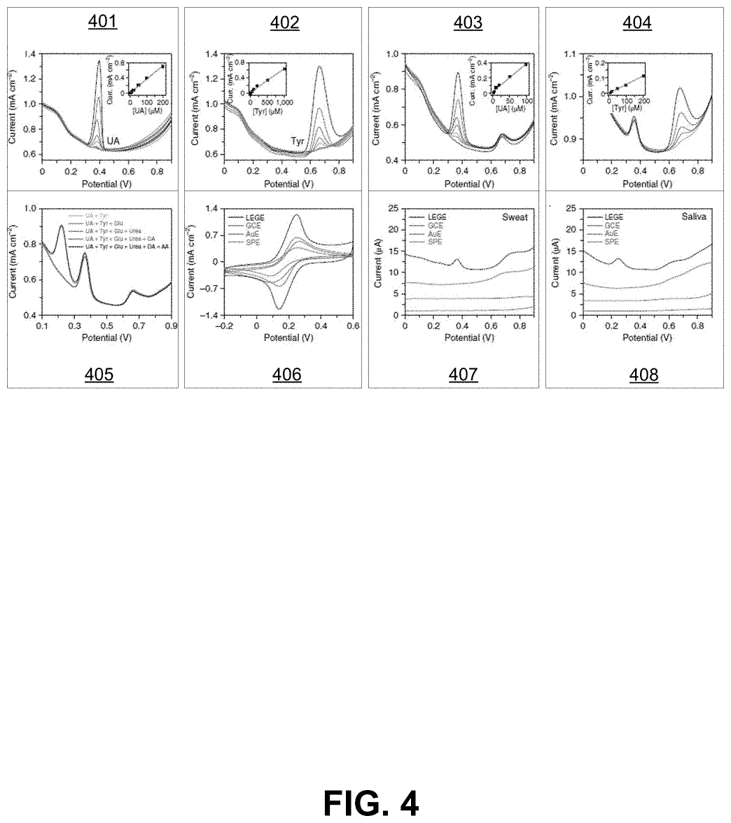

FIG. 4 illustrates, by way of example, detection of a target molecule using systems and methods in accordance with various embodiments of the disclosure. Frames 401 and 403, for example, depict detection and quantification of uric acid, and the selective detection of uric acid in the presence of 50 μM tyrosine, respectively. Frames 402 and 404 depict detection and quantification of tyrosine, and the selective detection of tyrosine in the presence of 50 μM uric acid, respectively. Insets therein depict the corresponding calibration curves. Frame 405 depicts the selective detection of both uric acid and tyrosine simultaneously in the presence of multiple common interferences often found in sweat, including glucose, urea, dopamine (DA), and AA (ascorbic acid). Frames 406-408 compare cyclic voltammetry scans of a chemical sensor electrode based on the systems and methods disclosed herein (e.g., LEGE denotes laser engraved chemical sensor), vs. a glassy carbon electrode (e.g., GCE), and gold electrode (e.g., AuE) in various solutions.

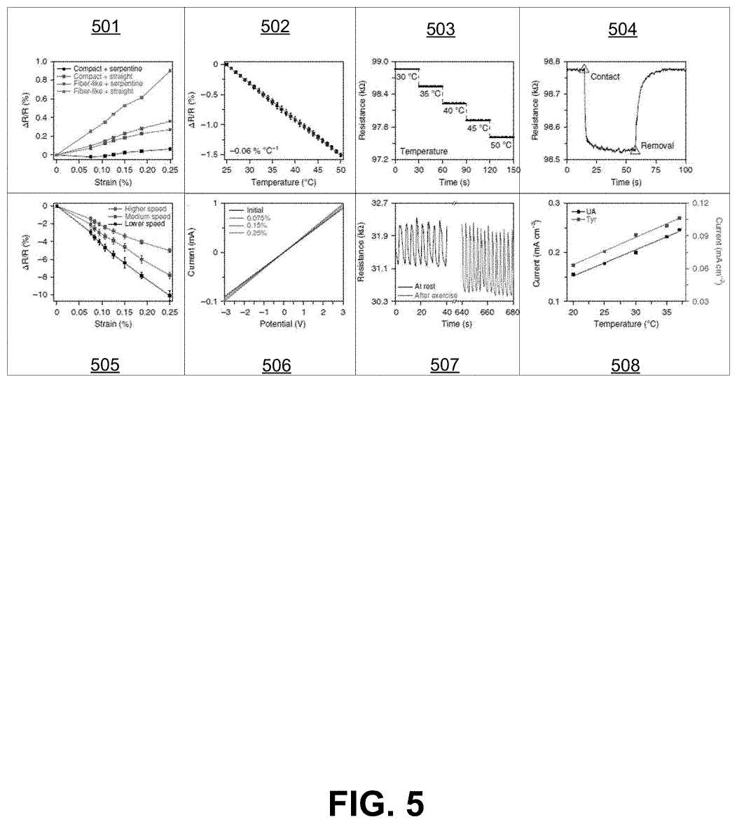

FIG. 5 depicts vital sign detection using systems and methods in accordance with various embodiments of the disclosure. Frames 501, 502, 505, and 506 depict, for example, multiple laser engraving parameters and calibrations that may be used during manufacturing of the resistive sensors that may comprise electrode 130. Dynamic detection of changes in physiological temperature over time (e.g., frame 503), temperature variation upon contact and removal from user (e.g., frame 504), and real-time respiration rate of a user at rest and after exercise (e.g., frame 507) are also depicted. Resistive sensor readings using various systems and methods disclosed herein may also be used to calibrate chemical sensor readings during on-body use (e.g., frame 508).

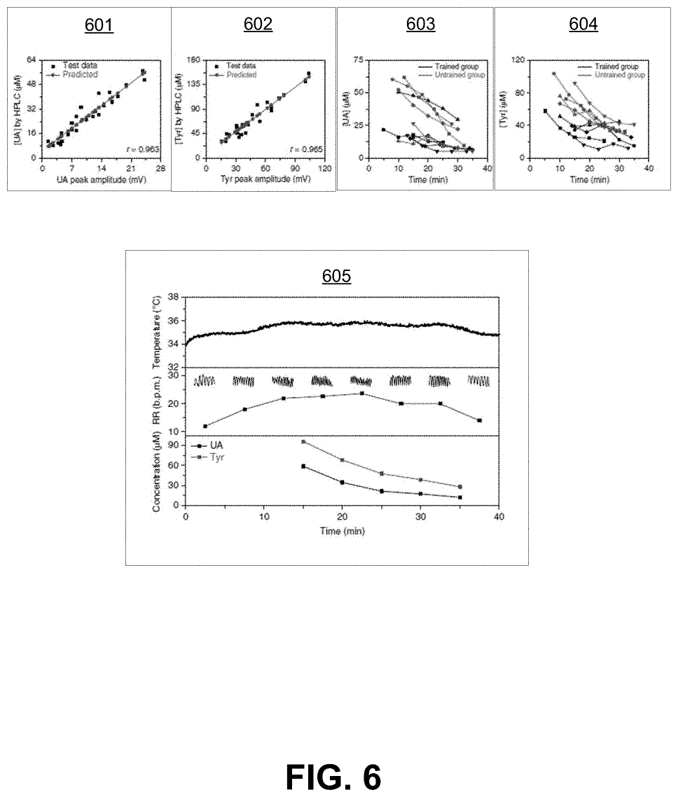

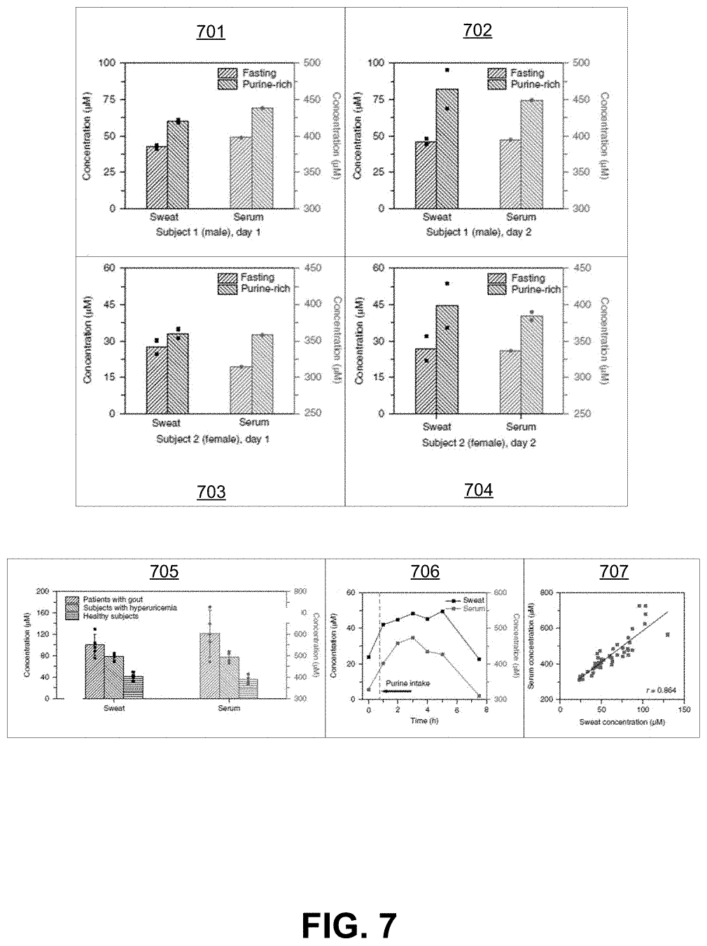

FIGS. 6 and 7 depict in-vivo validation of various systems and methods disclosed herein. In FIG. 6, continuous in-situ detection and quantification of a target molecule from raw sweat sampling including, for example, uric acid (e.g., frame 601) and tyrosine (e.g., frame 602), is validated against HPLC analysis of the same. Frames 603 and 604 depict, by way of example, dynamic monitoring of uric acid and tyrosine in physically trained and untrained human subjects during controlled cycling exercises. Frame 605 depicts, by way of example, continuous in-situ monitoring of respiration rate (“RR”, measured in breaths per minute), temperature, and sweat UA and Tyr levels from the neck of a healthy subject during constant-load stationary cycling. In FIG. 7, uric acid, for example, is detected in the sweat of multiple subjects over a period of two days (e.g., frames 701-704) using systems and methods in accordance with various embodiments of the disclosure. Frame 705 depicts, for example, sensor sensitivity for monitoring levels of uric acid in the sweat of patients diagnosed with gout and hyperuricemia. Dynamic detection of a target molecule (for example, uric acid) before and after dietary changes over a 7-hr period is shown in frame 706, while the Pearson correlation (R=0.867) between sweat and serum levels of the same target is shown in frame 707.

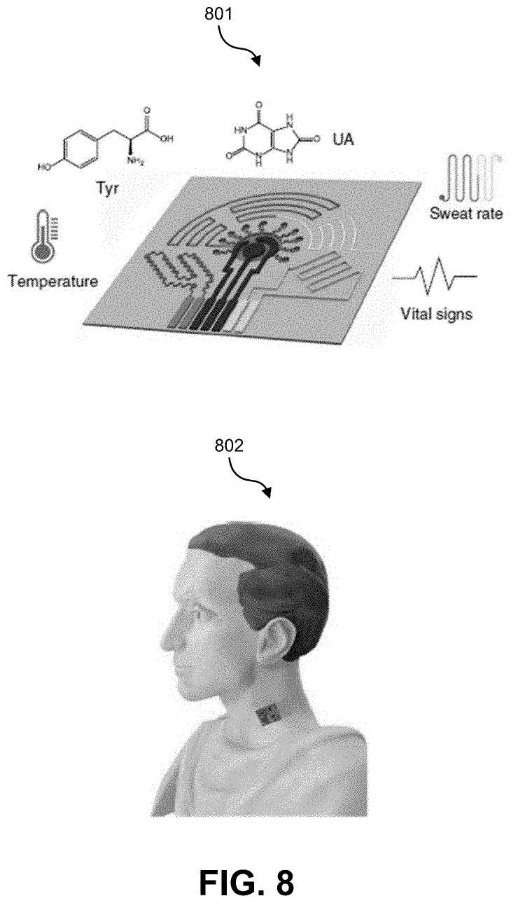

FIG. 8 illustrates, by way of example, the various sensing capabilities of a multimodal sensing layer as it may be used in accordance with various systems and methods of the disclosure. Multimodal sensing 801 may comprise, for example, multiple electrodes including a chemical sensor, a resistance temperature sensor, and a piezoresistive sensor. The chemical sensor may be used to detect a target molecule, or multiple target molecules simultaneously including for example, tyrosine and uric acid; the resistance temperature sensor may be used to detect, for example, the body temperature of a user; and the piezoresistive sensor may be used to detect various vital signs of the user including, for example, heartrate and respiration rate. Due to its compact, flexible, and lightweight structure, lab-on-skin biosensor 802 may be worn on various body parts of the user, including, for example, on the neck, arm, chest, back, and/or forehead of the user.

Referring again to FIG. 1, lab-on-skin biosensor 100 may also include a moisture resistant layer 140. In embodiments, moisture resistant layer 140 is coupled to the multifluidics layer 110. In some embodiments, moisture resistant layer 140 is coupled to the first surface of the microfluidics layer 110. In some embodiments, moisture resistant layer 140 may be used to prevent too much biological sample from reaching the microfluidics layer 110, including, for example, to control the flow rate of the biological sample. In some embodiments, moisture resistant layer is positioned between the skin surface of a user and the microfluidics layer 110 to prevent a portion of a biological sample from entering the microfluidics layer 110. In some embodiments, moisture resistant layer 140 is coupled to the first surface of microfluidics layer 110 and may comprise a laser engraved aperture, for example, to enable a portion of the biological sample enter the microchannels of microfluidics layer 110. In embodiments, moisture resistant layer 140 may comprise more than one laser engraved apertures through which a biological sample may enter the microchannels of microfluidics layer 110.

In embodiments, moisture resistant layer 140 may comprise polyethylene (PE). In some embodiments, moisture resistant layer 140 may comprise double-sided medical tape. Other plastics and materials may also be used to manufacture moisture resistant layer 140 including metals, glass, ceramics, and wood-based products. In some embodiments, the double-sided medical tape that is attached to the user also has at least one aperture to allow the passage of a biological sample from a user to the moisture resistant layer. It is to be understood that other adhesives may be used to attach the moisture resistant layer 140 both to the user and to the microfluidics layer 110. Such adhesives may include, for example, glue or other resins.

In embodiments, moisture resistant layer 140 may be coupled to microfluidics layer 110 through adhesive attachment. For example, moisture resistant layer 140 may be coupled to microfluidics layer 110 through double-sided medical tape, such that the double-sided medical tape is sandwiched between moisture resistant layer 140 and microfluidics layer 110. In some embodiments, the double-sided medical tape may have inlets and channels laser engraved into its surface to allow the passage of a biological sample from the moisture resistant layer 140 to the microfluidics layer 110. In some embodiments, double-sided medical tape is attached to both sides of moisture resistant layer 140, such that the moisture resistant layer 140 may simultaneously attach to a user and microfluidics layer 110.

Referring still to FIG. 1, lab-on-skin biosensor 100 may also include a logic circuit 150. In embodiments, logic circuit 150 may be electrically coupled to electrode 130 and may include a processor and a non-transitory memory with computer executable instructions embedded thereon. In various embodiments, logic circuit 150 may also include other circuits receiving, processing, and/or storing content, data, and other information. Logic circuit 150 may also, for example, facilitate the receipt of such content, data, or other information, as well as the generation of such content, data, or other information by the lab-on-skin biosensor 100. In embodiments, logic circuit 150 may comprise a flexible printed circuit board (FPCB).

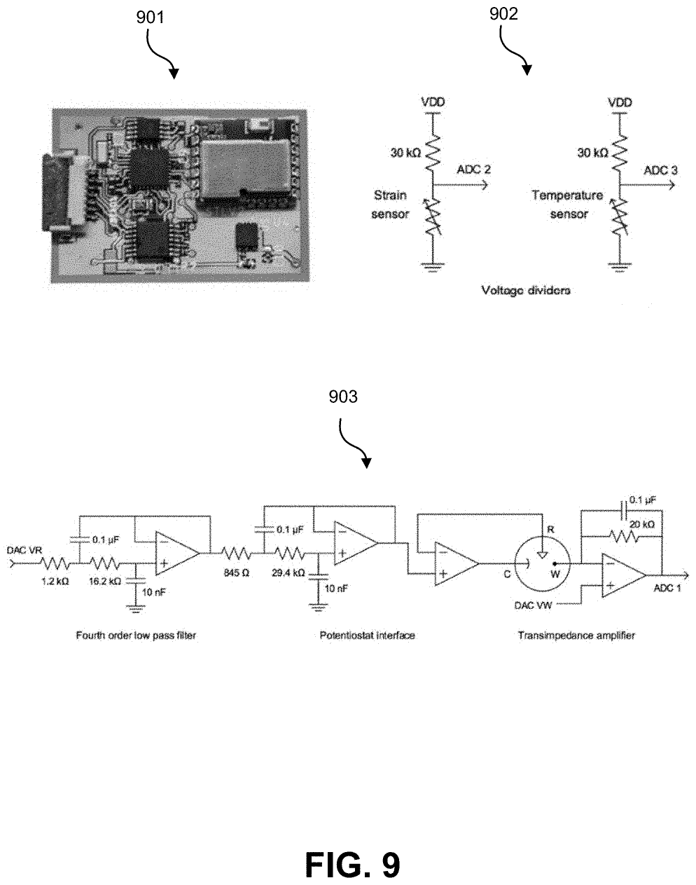

FIG. 9 illustrates, by way of example, circuitry that be implemented by a lab-on-skin biosensor, in accordance with various embodiments of the disclosure. For example, flexible printed circuit board (FPCB) 901 may be used by the systems and methods disclosed herein. Circuit diagram 902 depicts, for example, voltage dividers for driving resistive sensor measurements. Circuit diagram 903 depicts, by way of example, analog front-end potentiostat for voltammetric sensing (WR and VW correspond to the reference potential and working potential, respectively).

In embodiments, the computer executable instructions embedded within logic circuit 150 cause the processor to identify the electrical property detected with electrode 130. In some embodiments, the computer executable instructions embedded within logic circuit 150 cause the processor to identify the electrical property detected with electrode 130 when the target molecule is present in the biological sample. In some embodiments, the computer executable instructions embedded within logic circuit 150 cause the processor to identify the electrical property detected with electrode 130, wherein the electrical property is a change in temperature, strain, pressure, and/or resistance.

In some embodiments, the computer executable instructions embedded within logic circuit 150 cause the processor to generate an indication identifying the presence of the target molecule based on the electrical property detected by electrode 130. In some embodiments, the computer executable instructions embedded within logic circuit 150 cause the processor to generate an indication of a detected electrical property by electrode 130. In some embodiments, an electrical property may include, for example, voltage, amplitude, temperature, and/or resistance.

In embodiments, the indication generated by the processor may be transmitted electrically to a display to be identified visually. In some embodiments, the indication may be transmitted electrically to an LED, or a plurality of LEDs, to be identified visually. In embodiments, the indication generated may be stored on the non-transitory memory of logic circuit 150. In some embodiments, the indication generated may be transmitted wirelessly to another electronic device. For example, the indication generated may be transmitted wirelessly via Bluetooth or over Wi-Fi.

Lab-on-skin biosensor 100 may include a display 160. In embodiments, display 160 displays the presence of a target molecule and may be electrically coupled to logic circuit 130. In some embodiments, upon the binding of a target molecule, the computer executable instructions cause the processor to generate and output an indication identifying the presence of the target molecule to display 160. In some embodiments, display 160 displays the temperature, respiration rate, and/or heartrate of the user. In some embodiments, upon the detection of a temperature, respiration rate, and or heartrate of a user, the computer executable instructions cause the processor to generate and output an indication identifying the physical measurement to display 160. Non-limiting examples of display 160 include: a liquid-crystal display (LCD); an organic LCD (OLCD); a light emitting diode display (LED); an organic light emitting diode display (OLED); digital light processing display (DLP); among others.

In various embodiments, lab-on-skin biosensor 100 may also include a uniform redox probe. A uniform redox probe may be used according to various embodiments disclosed herein to detect both electroactive and non-electroactive target molecules. Unlike electroactive target molecules, non-electroactive target molecules do not transfer their electrons (i.e., do not become oxidized). Uniform redox probe may be used to detect both electroactive and non-electroactive molecules indirectly through loss of current as a result of their concentration in a biological sample. In some embodiments, the uniform redox probe may be deposited on a surface of electrode 130. In some embodiments, the uniform redox probe may include ferric ferrocyanide (e.g., Prussian Blue). Non-limiting examples of a uniform redox probe that may be used include, for example, thionine; anthraquinone 2-carboxylic acid; ferrocenecarboxylic acid; tris(2,2′-bipyridine-4,4′-dicarboxylic acid)cobalt(III); among others.

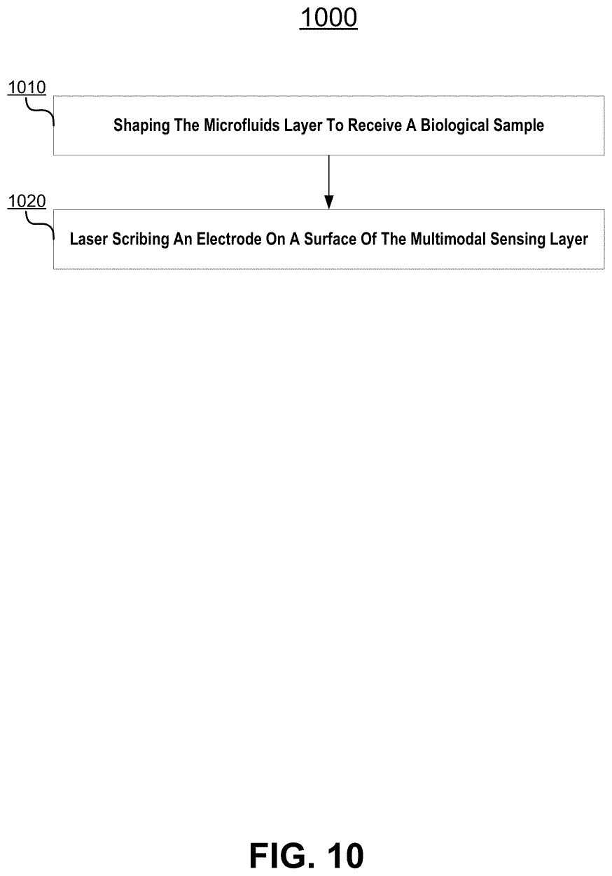

FIG. 10 is a flow diagram illustrating an example method in accordance with the technology disclosed. At a high level, method 1000 may be performed to manufacture a lab-on-skin biosensor in accordance with various embodiments of the disclosure. The operations of the various methods described herein are not necessarily limited to the order described or shown in the figures, and one of skill in the art will appreciate, upon studying the present disclosure, variations of the order of the operations described herein that are within the spirit and scope of the disclosure. Let it be appreciated that operations of method 1000 may be performed multiple times.

The operations and sub-operations of method 1000 may be carried out, in some cases, using one or more of the components, elements, devices, and sub-components of lab-on-skin biosensor 100, as described with respect to at least FIGS. 1-9, as well as components, elements, devices, and sub-components, depicted therein and/or described with respect thereto.

In such instances, the description of method 1000 may or may not refer to a corresponding component and/or element, but regardless of whether an explicit reference is made, one of skill in the art will recognize, upon studying the present disclosure, when the corresponding component and/or element may be used. Further, it will be appreciated that such references do not necessarily limit the described method to the particular component and/or element referred to. Thus, it will be appreciated by one of skill in the art that aspects and features described above in connection with (sub-) components, elements, devices, and components, including variations thereof, may be applied to the various operations described in connection with method 1000 without departing from the scope of the present disclosure.

Referring now to FIG. 10, method 1000 may be used for manufacturing lab-on-skin biosensor 100, in accordance with implementations of the disclosure. At operation 1010, microfluidics layer 110 is shaped to receive a biological sample. In embodiments, shaping microfluidics layer 110 may comprise laser engraving a microchannel and/or inlet into a surface of microfluidics layer 110. In some embodiments, more than one microchannel and/or inlet may be laser engraved into a surface of microfluidics layer 110. In some embodiments, shaping microfluidics layer 110 may comprise laser engraving multiple microchannels and/or inlets into microfluidics layer 110 such that the microchannels and/or inlets are transversely oriented to channel a biological sample from a first surface of the microfluidics layer 110 to a second surface of microfluidics layer 110. In embodiments, shaping microfluidics layer 110 may also comprise laser engraving a reservoir into microfluidics layer 110. In some embodiments, shaping microfluidics layer 110 may also comprise laser engraving more than one reservoir into microfluidics layer 110. In some embodiments, shaping microfluidics layer 110 may comprise laser engraving in vector mode. In some embodiments, shaping microfluidics layer 110 may comprise laser engraving in raster mode.

In embodiments, operation 1020 may include laser scribing electrode 130 on a surface of multimodal sensing layer 120. In some embodiments, one electrode 130 may be laser scribed on a surface of multimodal sensing layer 120. In some embodiments, more than one electrode 130 may be laser scribed on a surface of multimodal sensing layer 120. In embodiments, laser scribing electrode 130 on a surface of multimodal sensing layer 120 may comprise laser scribing or engraving by vector mode. In some embodiments, laser scribing electrode 130 on a surface of multimodal sensing layer 120 may comprise laser scribing or engraving by raster mode.

Different parameters may be set for the laser including, for example, laser speed, laser mode, and laser power, depending on the type of laser used and the type of sensor being manufactured that comprises electrode 130. For example, electrode 130 may comprise a chemical sensor. In instances where electrode 130 is a chemical sensor, the laser engraving/scribing parameters that may be used to manufacture electrode 130 may include, for example, engraving in raster mode, at 3.15 W power, speed 5.5%, and 1000 pulses per inch (PPI). In instances where electrode 130 is a resistive sensor (either temperature or piezoresistive), laser based fabrication may be performed in vector mode to allow for single continuous straight lines and/or serpentine lines. In various embodiments, multimodal sensing layer 120 comprises polyimide. In instances where multimodal sensing layer 120 comprises polyimide, laser scribing/engraving an electrode on the surface of multimodal sensing layer 120 results in electrode 130 comprising graphene.

Lab-on-skin biosensor 100 of FIG. 1 may be used to detect a target molecule in a biological sample. In embodiments, a biological sample may include an excreted bodily fluid, such as, for example, sweat, urine, tears, blood, salvia, and secretions from the male and female sex organs. Target molecules may include proteins (including viral proteins), antibodies, electrolytes, vitamins, amino acids, metabolized drugs, among other molecules and/or compounds. In some embodiments, a target molecule may include an electroactive molecule. Electroactive molecules may include, for example, tryptophan, tyrosine, phenylalanine, dopamine, vitamin C, vitamin B6, vitamin B12, uric acid, mycophenolic acid, caffeine, methionine, cortisol, noradrenaline, or adrenaline. In embodiments, a target molecule may comprise a non-electroactive molecule. Non-electroactive molecules may include, for example, leucine, iso-leucine, valine, busulfan, cyclophosphamide, creatinine, or urea. The lists of electroactive and non-electroactive molecules are not meant to be exhaustive. It is to be understood that additional electroactive/non-electroactive molecules not listed here, may also be detected according to the various systems and methods disclosed herein.

In various embodiments, a target molecule may include an amino acid. Amino acids that may be detected using embodiments of the disclosure include: alanine; glycine; isoleucine; leucine; proline; valine; phenylalanine; tryptophan; tyrosine; aspartic acid; glutamic acid; arginine; histidine; lysine; serine; threonine; cysteine; methionine; asparagine; and glutamine.

In various embodiments, a target molecule may include antibodies against viral nucleocapsid proteins or other virus-specific identifiers (i.e., epitope). For example, monoclonal antibodies against the SARS-CoV-2 nucleocapsid protein may be detected. Other monoclonal antibodies that are designed to detect other epitopes of the virus or other viruses may also be used as a target molecule. In some embodiments, other virus-specific target molecules, including molecules secreted by the virus, building block molecules of the virus, and genetic elements of the virus may also be a target molecule and be detected using technology disclosed herein.

In various embodiments, a target molecule may include a vitamin or provitamin (i.e., vitamin precursors). For example, vitamins and provitamins that may be detected include: thiamine (vitamin B1); riboflavin (vitamin B2); niacin (vitamin B3); choline (vitamin B4); pantothenic acid (vitamin B5); pyridoxine (vitamin B6); biotin (vitamin H, vitamin B7, or vitamin B8); folic acid (vitamin B9 or folate); and cobalamin (vitamin B12); ascorbic acid (vitamin C); retinol (vitamin A); calciferol (vitamin D); tocopherol (vitamin E); phylloquinone (vitamin K1); menaquinone (vitamin K2); β-carotin (vitamin A); 7-dehydrocholesterol (vitamin D); and cholecalciferol (vitamin D).

In various embodiments, the target molecule may include, for example, a hormone. Hormones that may be detected include: cholesterol; cortisol; progesterone; testosterone; corticosterone; aldosterone; β-estradiol; insulin; estrogen; thyroxin; gonadotropin-releasing hormone (GnRH); corticotropin-releasing hormone; melatonin; human growth hormone (HGH); adrenocorticotropic hormone; prolactin; and angiotensin. In some embodiments, a target molecule may include a protein used for diagnosis purposes, including for detecting and monitoring various illnesses (e.g., cancer). For example, a target molecule may include tumor markers for detecting and monitoring cancer, including: serum carcinoembryonic antigen (CEA); serum lipid-associated sialic acid (LASA); serum cancer antigen 19-9 (CA 19-9); cancer antigen 125 (CA 125); alpha fetoprotein (AFP); lactase dehydrogenase (LDH); and human chorionic gonadotropin (hCG). The list of target molecules is not meant to be exhaustive. It is to be understood that additional target molecules not listed here, may also be detected according to the various systems and methods disclosed herein.

By utilizing lab-on-skin biosensor 100 in accordance with the technology disclosed herein may be used to detect and measure quantity of a target molecule in a biological sample, and/or determine the vital signs of a user. For example, medical, veterinary, research staff, law enforcement, or other interested personnel can use the disclosed technology to detect the presence and/or measure the quantity of a target molecule in a biological sample. Furthermore, the same interested personnel may use the disclosed technology to determine certain vital signs and/or temperature of a subject wearing lab-on-skin biosensor 100. By identifying certain target molecules including, for example, drug metabolites, and/or vital signs, including respiratory rate and heartrate, interested personnel can determine if a subject (e.g., a human, animal, or organism) has taken a certain drug and/or may observe whether a subject is in compliance in taking prescription medications. Moreover, by identifying certain target molecule, including certain metabolites and amino acids, and by identifying certain vital signs, an interested personnel can determine if a subject is experiencing a certain medical issue, or diagnose a certain medical issue. Embodiments of the technology disclosed herein enable analysis locally at the lab-on-skin biosensor without the need for separate equipment, resulting in a less complex system that is smaller and portable. This makes it easier for interested personnel and subjects to view the biosensor data at the device, eliminating the need to utilize other equipment, e.g., enabling field tests for detection of illicit drugs in a subject, or compliance with a drug regiment by the subject. In some embodiments, a portable biosensor may include a built-in USB connector, enabling the portable monitoring device to be directly attached to a computer after use to store or review data, or (as discussed above) to charge the portable biosensor.

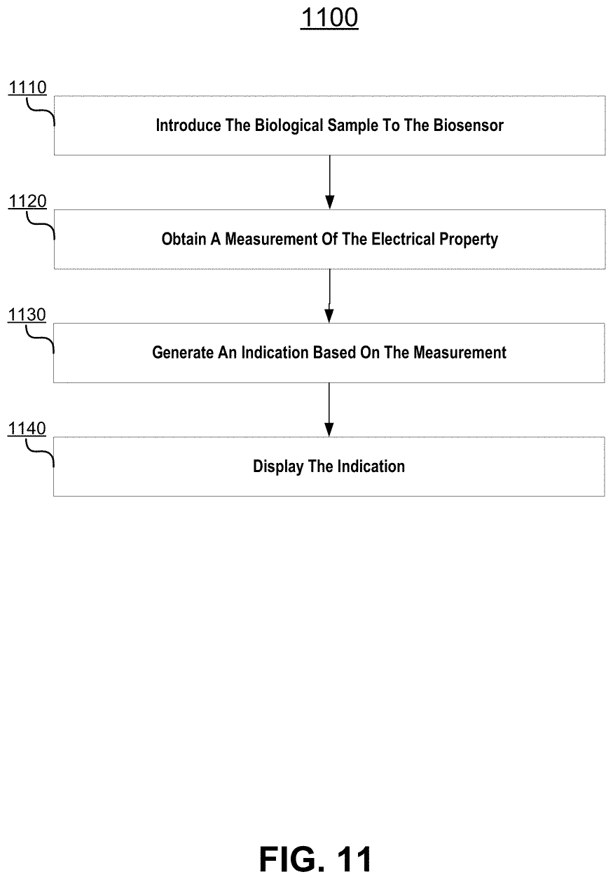

FIG. 11 is a flow diagram illustrating an example method in accordance with the technology disclosed. At a high level, method 1100 may be performed to identify/quantify a target molecule in a biological sample and to determine multiple vital signs of a subject using lab-on-skin biosensor 100. The operations of the various methods described herein are not necessarily limited to the order described or shown in the figures, and one of skill in the art will appreciate, upon studying the present disclosure, variations of the order of the operations described herein that are within the spirit and scope of the disclosure. Let it be appreciated that operations of method 1100 may be performed multiple times.

The operations and sub-operations of method 1100 may be carried out, in some cases, by and/or using one or more of the components, elements, devices, and sub-components of lab-on-skin biosensor 100, as described with respect to at least FIGS. 1-10, as well as components, elements, devices, and sub-components, depicted therein and/or described with respect thereto.

In such instances, the description of method 1100 may or may not refer to a corresponding component and/or element, but regardless of whether an explicit reference is made, one of skill in the art will recognize, upon studying the present disclosure, when the corresponding component and/or element may be used. Further, it will be appreciated that such references do not necessarily limit the described methods to the particular component and/or element referred to. Thus, it will be appreciated by one of skill in the art that aspects and features described above in connection with (sub-) components, elements, devices, and components, including variations thereof, may be applied to the various operations described in connection with method 1100 without departing from the scope of the present disclosure.

Referring now to FIG. 11, method 1100 may be used for detecting and quantifying a target molecule in a biological sample, and for determining multiple vital signs of a person using lab-on-skin biosensor 100, in accordance with implementations of the disclosure. At operation 1110, a biological sample that may include a target molecule is introduced to lab-on-skin biosensor 100. In embodiments, the biological sample may be introduced to lab-on-skin biosensor 100 when the biological sample is collected by the microfluidics layer 110. For example, where the biological sample is sweat, the biological sample may be collected by microfluidics layer 110 directly from the skin of the wearer. Where the biological sample is saliva, for example, the biological sample may be collected from the wearer's mouth or through a tube or other device that funnels saliva to the microfluidics layer 110. Other, non-limiting ways the biological sample may be collected by microfluidics layer 110 include through pipetting, syringe injection, column feeding, micro-pumping, and various machine-automated methods.

Several different biological samples including, for example, blood, sweat, tears, urine, saliva, and/or breath condensation (e.g., condensed vapor) may be introduced to lab-on-skin biosensor 100 in order to detect a target molecule. In embodiments, the target molecule of the biological sample may include an electroactive target molecule. In some embodiments, the target molecule of the biological sample may include a non-electroactive target molecule. In some embodiments, more than one target molecule may be in the biological sample and introduced to the biosensor. In embodiments, the target molecule may include electroactive and/or non-electroactive target molecules.

At operation 1120, electrode 130 of lab-on-skin biosensor 100 may obtain a measurement of the electrical property of a target molecule present in a biological sample collected and funneled to the multimodal sensing layer 120 by microfluidics layer 110, in accordance with various embodiments of the disclosure. In embodiments, electrode 130 may detect a measurement of an electrical property of target molecule present in the biological sample collected by microfluidics layer 110. In some embodiments, electrode 130 may detect a measurement of an electrical property of a physical event (e.g., strain, pressure, resistance, and/or temperature). In embodiments, detection of an electrical property of a target molecule with electrode 130 may include the passing of an electrolyte from the target molecule to electrode 130. In embodiments, detection of an electrical property of a target molecule with electrode 130 may include the inhibition of the passing of an electrolyte from the target molecule to electrode 130. In some embodiments, detection of an electrical property of a physical event may include detection, or the inhibition in detecting, electron flow variation due to temperature, strain, and/or pressure of the user.

In embodiments, the electrical property detected by electrode 130 may include an electrical current. In some embodiments, the electrical property may include an electrical voltage. In some embodiments, the electrical property may include an electrical impedance. In some embodiments, the electrical property may include one or more of an electrical current, voltage, and/or impedance. In embodiments, a measurement of an electrical property may include reaching or meeting a threshold of an electrical property. In some embodiments, a measurement of an electrical property may include reaching or meeting a threshold on an electrical property such that when the threshold is reached or met, the measurement may be recorded and sent to the logic circuit for processing. In embodiments, a measurement may include a change in the electrical property. In some embodiments, a measurement may include a change in the electrical property such that when the change in electrical property is detected, for example, from a baseline, the measurement may be sent to the logic circuit for processing. In some embodiments, a change in electrical property may include an increase or decrease in the electrical property from a certain baseline.

In embodiments, more than one measurement of an electrical property may be obtained by sweeping electrode 130 such that the multimodal sensing layer 120 may be regenerated during a reading. In some embodiments, sweeping electrode 130 allows for continuous detection of a measurement of an electrical property. In some embodiments, sweeping electrode 130 may allow for continuous detection of more than one measurement of an electrical property. Sweeping electrode 130 may include rapid voltammetric and/or amperometric sweeping.

Referring still to FIG. 11, at operation 1130, logic circuit 150 may generate an indication based on the measurement detected by electrode 130, in accordance with embodiments of the disclosure. In embodiments, logic circuit 150 may include a processor and a non-transitory memory with computer executable instructions embedded thereon. In embodiments, electrode 130 may be electrically coupled to logic circuit 150, and upon the detection of a target molecule, the computer executable instructions cause the processor to identify the electrical property detected with electrode 130. In some embodiments, electrode 130 may be electrically coupled to logic circuit 150, and upon a vital sign threshold measurement (e.g., certain temperature, respiration rate and/or heartrate), the computer executable instructions cause the processor to identify the certain measurement detected with electrode 130. In some embodiments, the computer executable instructions cause the processor to generate an indication identifying the presence of the target molecule based on the electrical property detected with electrode 130. In some embodiments, the computer executable instructions cause the processor to generate an indication identifying the vital sign (including a certain threshold, or its presence or absence) based on the measurement detected with electrode 130. The indication generated may be identified visually by a display and/or LED(s), through other means of sensory communication including auditory cues, or the indication generated may be wirelessly transmitted via Bluetooth or WiFi to a remote component.

In various embodiments, at operation 1140, logic circuit 150 may output the indication identifying the presence of the target molecule to display 160 in accordance with various embodiments of the disclosure. In some embodiments, at operation 1140, logic circuit 150 may output the indication identifying a vital sign of the user to display 160 in accordance with various embodiments of the disclosure. In some embodiments, upon the detection of the measurement of an electrical property of a target molecule with electrode 130, the computer executable instructions of logic circuit 150 further cause the processor to output the indication identifying the presence of the target molecule to display 160. In some embodiments, upon the detection of the measurement of a vital sign of a user with electrode 130, the computer executable instructions of logic circuit 130 further cause the processor to output the indication identifying the measurement of the vital sign to display 160. In embodiments, display 160 may display visually the indication identifying the presence of the target molecule and/or the measurement of the vital sign. In some embodiments, the display may include an LCD screen. In some embodiments, the indication displayed may include a visual representation of the measurement of the electrical property, including, for example, an electrical current and/or an electrical voltage. In some embodiments, the indication displayed may include a measurement of a vital sign, including for example, heartrate, respiration rate, and/or temperature of the user. In some embodiments, the indication displayed may include a visual representation of the measurement of the electrical property, including for example, a change in the electrical current and/or voltage. In some embodiments, the indication displayed may include a visual representation of the measurement of the electrical property, including for example, a change in the electrical impedance. In some embodiments, the visual representation may include, for example, a graph having an x and y-axis. In some embodiments, the indication may include a quantification of the amount of target molecule present in the biological sample. In some embodiments, the indication may include a change in measurement of a vital sign, including for example, change in temperature, change in respiration rate, and change in heartrate. In some embodiments, the quantification of target molecule present in a biological sample may include units of potential (e.g., voltage or “V”), current (e.g., amps or “A”), and/or impedance (e.g., ohms or “Z”). In some embodiments, the vital sign measurement may include, for example, units of degrees (e.g., ° F. and/or ° C.) oxygen consumption (VO2, in mL/kg/min), and beats per minute (bpm). In some embodiments, various amounts of each of these units may include nano-units, micro-units, mili-units, and/or liter-units.

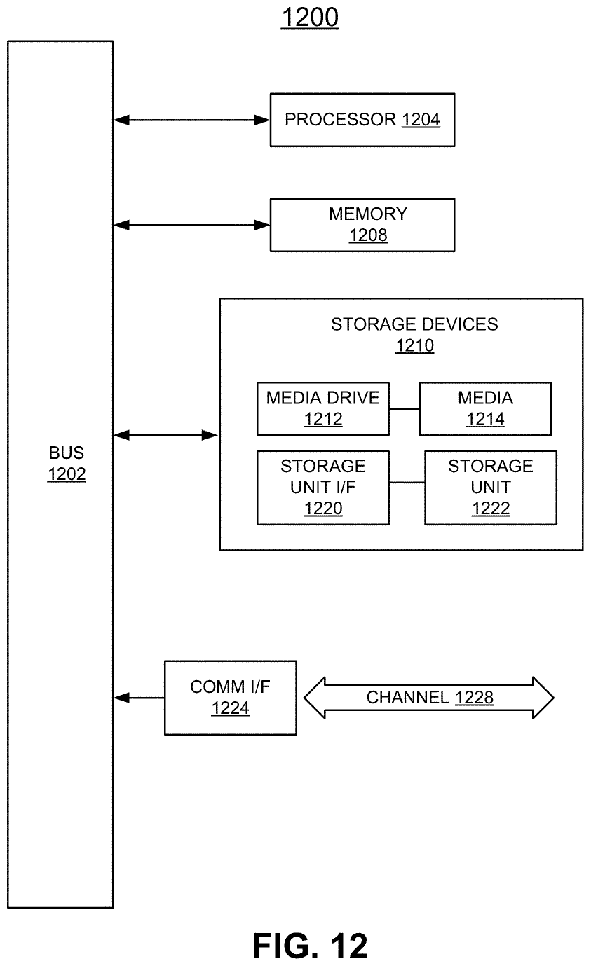

FIG. 12 illustrates example computing component 1200, which may, in some instances, include a processor/controller resident on a computer system (e.g., lab-on-skin biosensor 100). Computing component 1200 may be used to implement various features and/or functionality of embodiments of the systems, devices, and methods disclosed herein. With regard to the above-described embodiments set forth herein in the context of systems, devices, and methods described with reference to FIGS. 1 through 11, including embodiments involving lab-on-skin biosensor 100, one of skill in the art will appreciate additional variations and details regarding the functionality of these embodiments that may be carried out by computing component 1200. In this connection, it will also be appreciated by one of skill in the art upon studying the present disclosure that features and aspects of the various embodiments (e.g., systems) described herein may be implemented with respected to other embodiments (e.g., methods) described herein without departing from the spirit of the disclosure.

As used herein, the term component may describe a given unit of functionality that may be performed in accordance with one or more embodiments of the present application. As used herein, a component references a module, and/or may be implemented utilizing any form of hardware, software, or a combination thereof. For example, one or more processors, controllers, ASICs, PLAs, PALs, CPLDs, FPGAs, logical components, software routines or other mechanisms may be implemented to make up a component. In embodiment, the various components described herein may be implemented as discrete components or the functions and features described may be shared in part or in total among one or more components. In other words, as would be apparent to one of ordinary skill in the art after reading this description, the various features and functionality described herein may be implemented in any given application and may be implemented in one or more separate or shared components in various combinations and permutations. Even though various features or elements of functionality may be individually described or claimed as separate components, one of ordinary skill in the art will understand upon studying the present disclosure that these features and functionality may be shared among one or more common software and hardware elements, and such description shall not require or imply that separate hardware or software components are used to implement such features or functionality.

Where components of the application are implemented in whole or in part using software, in one embodiment, these software elements can be implemented to operate with a computing or processing component capable of carrying out the functionality described with respect thereto. One such example computing component is shown in FIG. 12. Various embodiments are described in terms of this example-computing component 1200. After reading this description, it will become apparent to a person skilled in the relevant art how to implement the application using other computing components or architectures.

Referring now to FIG. 12, computing component 1200 may represent, for example, computing or processing capabilities found within a self-adjusting display, desktop, laptop, notebook, and tablet computers; hand-held computing devices (tablets, PDA's, smart phones, cell phones, palmtops, etc.); workstations or other devices with displays; servers; or any other type of special-purpose or general-purpose computing devices as may be desirable or appropriate for a given application or environment. Computing component 1200 might also represent computing capabilities embedded within or otherwise available to a given device. For example, a computing component might be found in other electronic devices such as, for example navigation systems, portable computing devices, and other electronic devices that might include some form of processing capability.

Computing component 1200 might include, for example, one or more processors, controllers, control components, or other processing devices, such as a processor 1204. Processor 1204 might be implemented using a general-purpose or special-purpose processing engine such as, for example, a microprocessor, controller, or other control logic. In the illustrated example, processor 1204 is connected to a bus 1202, although any communication medium can be used to facilitate interaction with other components of computing component 1200 or to communicate externally.

Computing component 1200 might also include one or more memory components, simply referred to herein as main memory 1208. For example, preferably random access memory (RAM) or other static or dynamic memory, might be used for storing information and instructions to be executed by processor 1204. Main memory 1208 might also be used for storing temporary variables or other intermediate information during execution of instructions to be executed by processor 1204. Computing component 1200 might likewise include a read only memory (“ROM”) or other static storage device coupled to bus 1202 for storing static information and instructions for processor 1204.

The computing component 1200 might also include one or more various forms of information storage mechanism 1210, which might include, for example, a media drive 1212 and a storage unit interface 1220. The media drive 1212 might include a drive or other mechanism to support fixed or removable storage media 1214. For example, a hard disk drive, a solid state drive, a magnetic tape drive, an optical disk drive, a compact disc (CD) or digital video disc (DVD) drive (R or RW), or other removable or fixed media drive might be provided. Accordingly, storage media 1214 might include, for example, a hard disk, flash drive, an integrated circuit assembly, USB, magnetic tape, cartridge, optical disk, a CD or DVD, or other fixed or removable medium that is read by, written to or accessed by media drive 1212. As these examples illustrate, the storage media 1214 can include a computer usable storage medium having stored therein computer software or data.

In alternative embodiments, information storage mechanism 1210 might include other similar instrumentalities for allowing computer programs or other instructions or data to be loaded into computing component 1200. Such instrumentalities might include, for example, a fixed or removable storage unit 1222 and an interface 1220. Examples of such storage units 1222 and interfaces 1220 can include a program cartridge and cartridge interface, a removable memory (for example, a flash memory or other removable memory component) and memory slot, a PCMCIA slot and card, and other fixed or removable storage units 1222 and interfaces 1220 that allow software and data to be transferred from the storage unit 1222 to computing component 1200.

Computing component 1200 might also include a communications interface 1224. Communications interface 1224 might be used to allow software and data to be transferred between computing component 1200 and external devices. Examples of communications interface 1224 might include a modem or softmodem, a network interface (such as an Ethernet, network interface card, WiMedia, IEEE 802.XX or other interface), a communications port (such as for example, a USB port, IR port, RS232 port Bluetooth® interface, or other port), or other communications interface. Software and data transferred via communications interface 1224 might typically be carried on signals, which can be electronic, electromagnetic (which includes optical) or other signals capable of being exchanged by a given communications interface 1224. These signals might be provided to communications interface 1224 via a channel 1228. This channel 1228 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to transitory or non-transitory media such as, for example, memory 1208, storage unit 1220, media 1214, and channel 1228. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing component 900 to perform features or functions of the present application as discussed herein.

Although described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the application, whether or not such embodiments are described and whether or not such features are presented as being a part of a described embodiment. Thus, the breadth and scope of the present application should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The use of the term “component” does not imply that the components or functionality described or claimed as part of the component are all configured in a common package. Indeed, any or all of the various components of a component, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

The details of some embodiments of the systems and methods of the present disclosure are set forth in this description and in some cases, in other portions of the disclosure. Other features, objects, and advantages of the disclosure will be apparent to one of skill in the art upon examination of the present disclosure, description, figures, examples, and claims. It is intended that all such additional systems, methods, devices, features, and advantages be included within this description (whether explicitly or by reference), be within the scope of the present disclosure, and be protected by one or more of the accompanying claims.

### EXAMPLES

### Example 1: Fabrication of the Chemical Sensor

For sensor patterning, a PI film was attached onto a supporting substrate in a 50 W CO₂ laser cutter. The optimized parameters for the chemical sensor were Power 6.3%, Speed 5.5%, Points Per Inch (PPI) 1000, raster mode. After graphene electrodes were scribed, silver was electrodeposited onto one pattern to function as the reference electrode (RE) at −0.2 mA for 100 s using a plating solution containing 250 mM silver nitrate, 750 mM sodium thiosulfate, and 500 mM sodium bisulfate. The physical sensors had their contact pads scribed with the same parameters as the chemical sensors. For the active sensing area of the strain sensor, the optimized parameters were Power 0.3%, Speed 1.2%, PPI 400, vector mode; for the active sensing area of the temperature sensor, the optimized parameters were Power 1.5%, Speed 11%, PPI 1000, vector mode. All sensors were scribed at focused height.

Example 2: Characterization Chemical Sensors

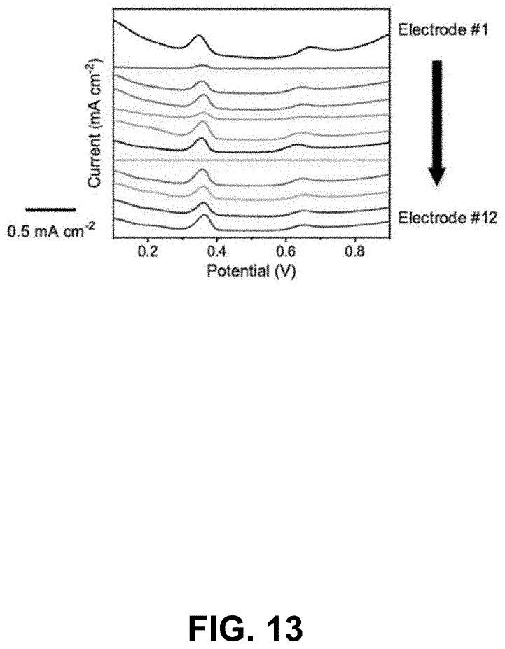

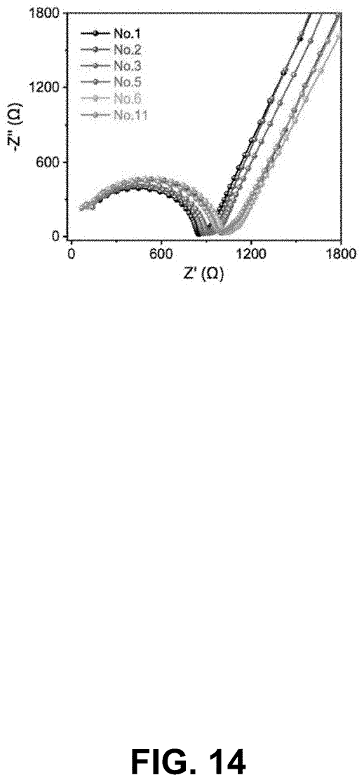

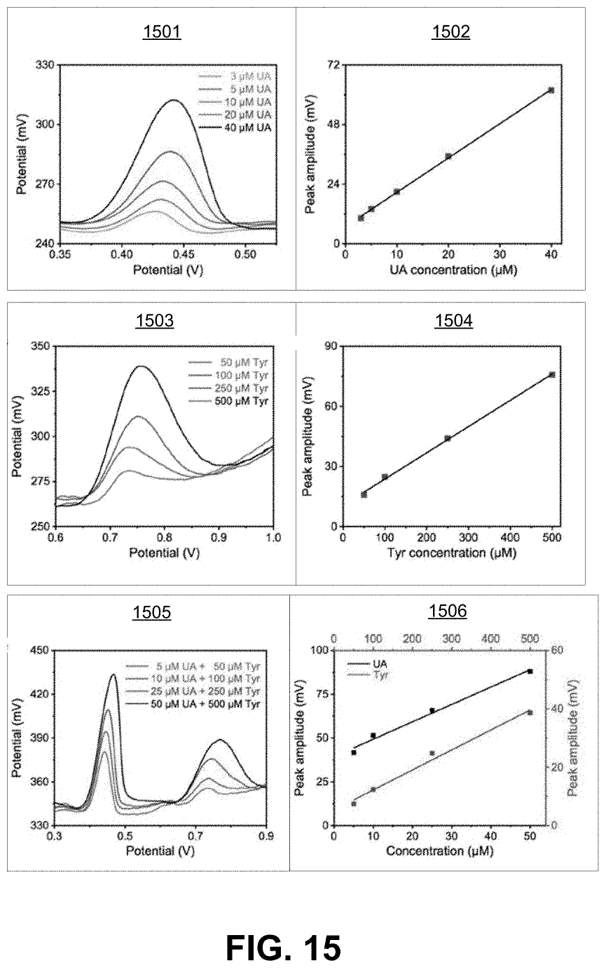

Highly sensitive chemical sensors are readily manufactured on PI via parallel lines of scans (raster mode). The three-electrode graphene sensor could selectively catalyze the oxidation of UA and Tyr at specific potentials (e.g., FIG. 3, frame 301). Based on the DPV peak amplitudes of UA and Tyr in the standard solutions (FIG. 13; Table 1, below) and electrochemical impedance spectroscopy (EIS) (FIG. 14), optimized laser engraving parameters (mode, raster; power, 3.15 W; speed, 5.5%; pulses per inch (PPI), 1000) are chosen to obtain the sensor with the best electrochemical catalytic performance. Various other target molecules may also be detected using the chemical sensors disclosed herein. For example, see FIG. 15, frames 1501, 1503, and 1505, with corresponding calibration curves of frames 1502, 1504, and 1506, respectively.

TABLE 1

| Electrode | Power | Speed | | UA Peak | Tyr Peak |
|---|---|---|---|---|---|
| # | (%) | (%) | PPI | (mA cm²) | (mA cm²) |
| 1 | 6.3 | 5.5 | 1000 | 0.142 | [symbol] |
| 2 | 5 | 5 | 1000 | [symbol] | 0 |
| 3 | 5 | 8 | 1000 | 0.091 | [symbol] |
| 4 | 8 | 7 | 1000 | 0.113 | [symbol] |
| 5 | 8 | 8 | 1000 | 0.06 | 0.013 |
| 6 | 5.5 | 5 | 1000 | 0 | 0 |
| 7 | 5.5 | 5 | 1000 | 0.132 | 0.043 |
| 8 | 7.5 | 8 | 1000 | [symbol] | [symbol] |
| 9 | 8 | 5 | 250 | 0.138 | 0.045 |
| 10 | 8 | 5 | 500 | [symbol] | [symbol] |
| 11 | 8 | 5 | 750 | 0.1 | 0.033 |
| 12 | 8 | 5 | 1000 | 0.131 | 0.031 |

[symbol]

indicates data missing or illegible when filed

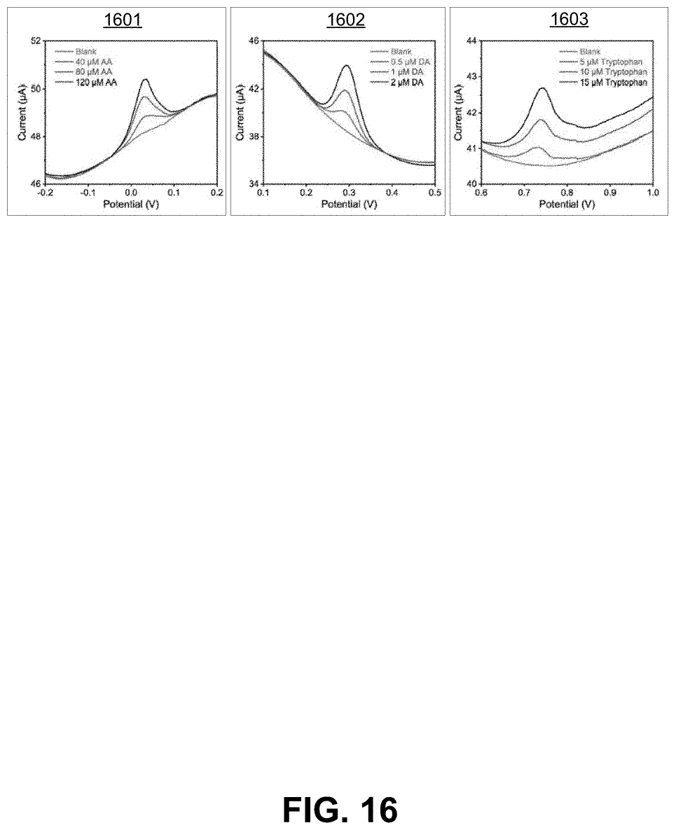

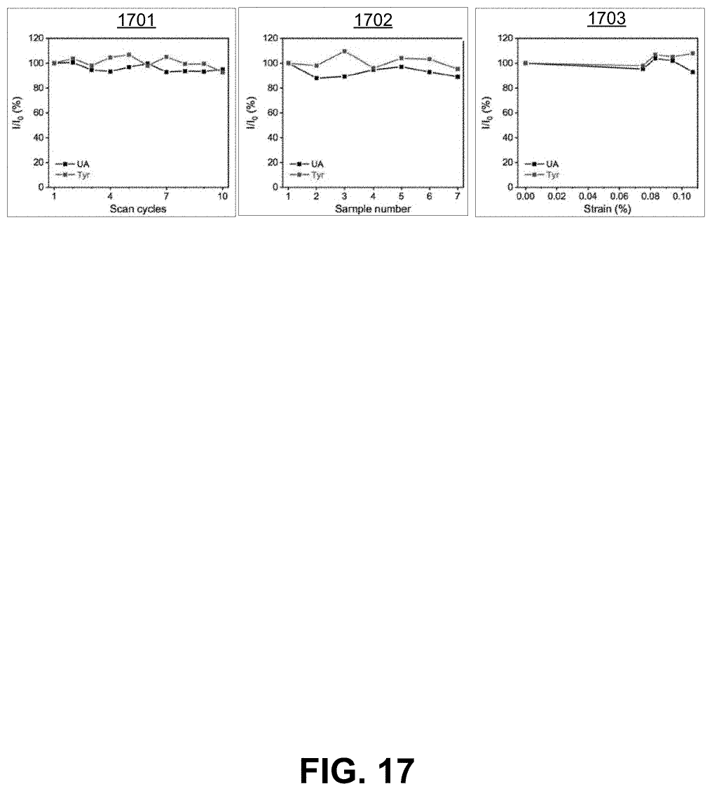

Chemical sensor characterizations were performed in 0.01 M acetate buffer saline (ABS) (pH 4.6 with the addition of 50 mM NaCl) unless otherwise noted. DPV analysis was performed through an electrochemical workstation (CHI 832D). The detailed parameters were: (1) range, 0-0.9 V; (2) incremental potential, 0.004 V; (3) pulse amplitude, 0.05 V; (4) pulse width, 0.05 s; (5) pulse period, 0.5 s; (6) sensitivity, le-5 A/V. 50 μM interference chemicals (glucose, urea, DA, and AA) were added to the solutions for the test in FIG. 4, at frame 405. The analytical performance of the chemical sensor towards AA, DA, and tryptophan detection was tested in ABS containing low concentrations of target analytes in FIG. 16, frames 1601-1603. The stability of the chemical sensor was evaluated by comparing the current during the successive scans or different solution drops in FIG. 17, frames 1701-1703. Analysis of mechanical stability of the chemical sensor was performed on 3D-printed curved molds (radii of bending curvature are 3.5, 4.0, 4.5, and 5 cm, respectively) (e.g., FIG. 17). The influence of temperature on the sensor performance was investigated by immersing the chemical sensor into an ABS solution containing 50 μM UA and 100 μM Tyr.

### Example 3: Fabrication of the Various Resistive Sensors

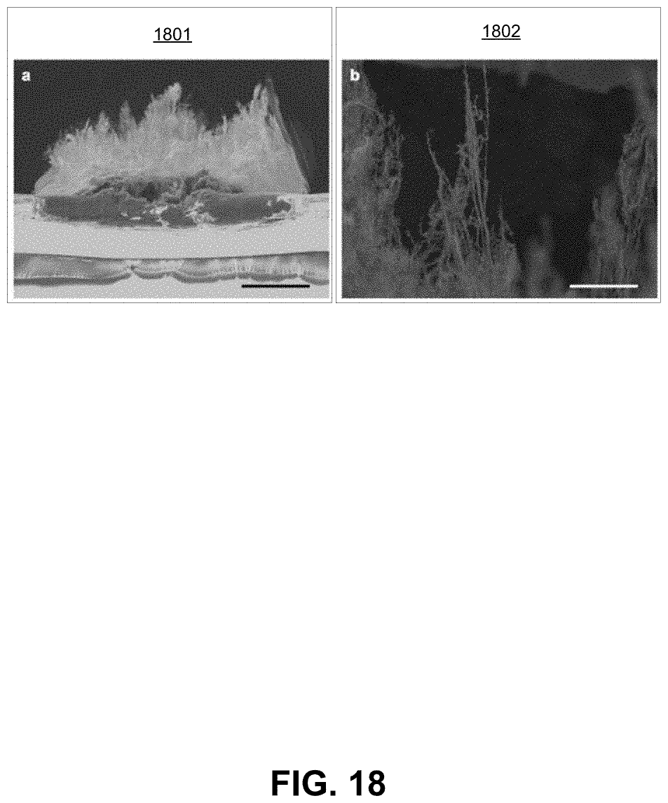

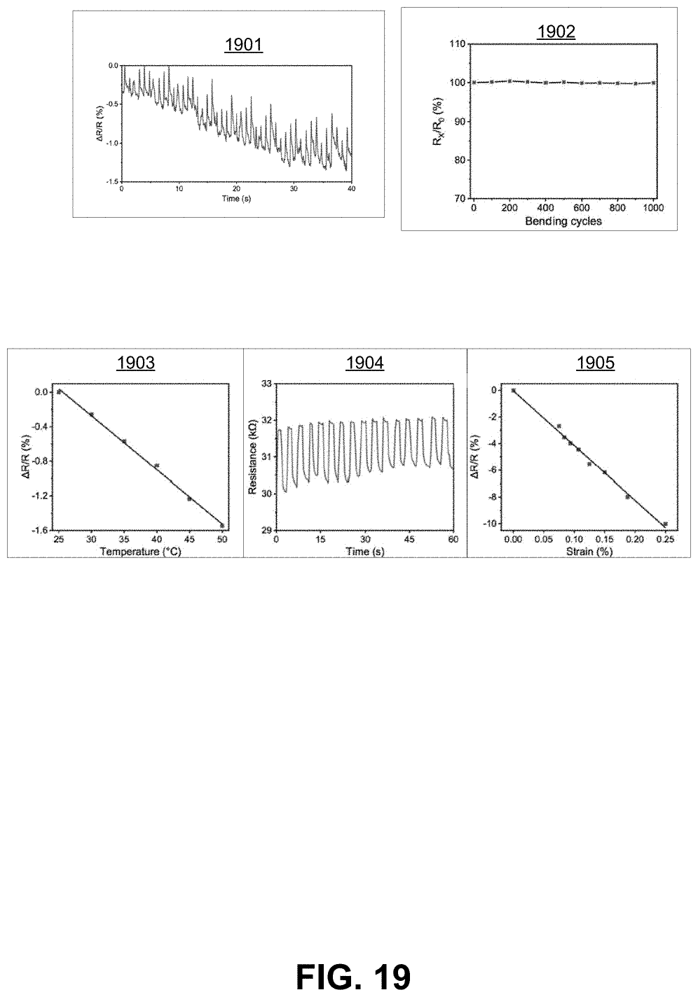

Resistive sensors (i.e., temperature and piezoresistive “strain” sensors) are fabricated in vector mode which engraves a single continuous line at a time (see FIG. 18, frames 1801 and 1802). Both material morphology and sensor layout play crucial roles to achieve the desired sensor performance: the fiber-like structure resulted from a high dose of local laser power (as illustrated in FIG. 18) coupled with the straight line design yields the highest strain response; the compact structure, coupled with serpentine line design is less susceptible to the strain variations. The former design was used for piezoresistive strain sensing, and the latter for temperature sensing to minimize the strain interference on temperature measurement. The temperature sensor shows a fast, accurate, and stable response to temperature variations with a sensitivity of −0.06% ° C.⁻¹ (see, FIG. 5, frames 502-504) which indicates the negative temperature coefficient (NTC) behavior of the sensor. The piezoresistive strain sensor is fabricated at a low laser speed for large strain response and high stability (frames 505 and 506), ideally suitable for dynamic monitoring of respiration rate (RR) (frame 507) and heart rate (HR) (FIG. 19, frame 1901). After 1000 bending cycles, the flat-state resistance of the strain sensor remains stable (frame 1902). The temperature and strain sensor response can be accurately monitored by the FPCB (frames 1903-1905).

### Example 4: Characterization of Various Resistive Sensors

The resistive temperature sensor characterization was performed on a ceramic hot plate (see FIG. 5, at frame 502 and 503). The sensor response was recorded using a parameter analyzer and compared with the readings from an infrared thermometer. Upon contact with and removal from the skin of a subject, the resistance of the temperature sensor was recorded (see FIG. 5, at frame 504). Bending studies were implemented on 3D-printed curved molds with varied radii. The response of the piezoresistive sensor was recorded using the parameter analyzer in a controlled temperature atmosphere (23±1° C.). To investigate the current responses under different strains, the I-V responses of the piezoresistive sensor were obtained under static strains (see FIG. 5, at frame 506). When attached on the neck and on the wrist, the piezoresistive sensor was able to monitor respiration rate and heart rate, respectively (See FIG. 5 at frame 507 and FIG. 19 at frame 1901). Under repetitive bending (radius of bending curvature, 3 cm) for 1000 cycles, the mechanical deformation effect on piezoresistive sensor was evaluated based on the resistance recorded every 100 cycles (See FIG. 19 at frame 1902).

### Example 5: Fabrication and Characterization of Microfluidic Channels

Double-sided medical adhesive tape was attached to a substrate in a laser cutter. One layer of medical adhesive was cut through to make the channels and the reservoir, and another layer of medical adhesive was used to interface skin with inlets. Between two layers lied a thin (12 μm) and transparent PET film (moisture resistant layer). The optimized laser cutter parameters were Power 1%, Speed 1.5%, PPI 1000 for reservoir outline and channels, and Power 2%, Speed 1%, PPI 1000 for inlet outlines, all in vector mode and at focused height. The sweat rates were measured via optical image analysis based on the photos of a microfluidic patch taken sequentially on the different body parts of the subjects. Estimated sweat rates were calculated by the sweat volume changes divided by the time intervals. For FIG. 2 frame 203, black dye was dropped in the reservoir, and a transparent 75 μm PET film was used instead of the PI layer to prepare the microfluidics for better visibility.

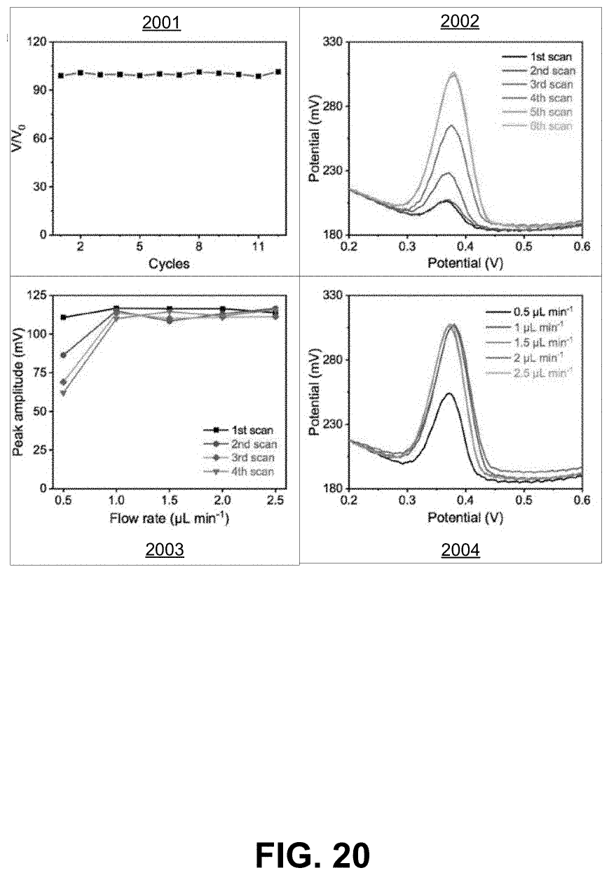

The lab-on-skin biosensor can reliably and continuously measure the UA levels (±0.44%) through successive DPV scans over a 12-cycle period (see FIG. 20, frame 2001). When the input solution is switched from 20 μM to 80 μM, the patch takes less than 3 minutes to reach new stable reading (see FIG. 20 at frame 2002), indicating the high temporal resolution of the microfluidic sensing system. To investigate if the depletion of the target molecules in the confined reservoir during DPV scans affects the sensing accuracy, wireless UA sensing is performed in a microfluidics layer with a varied flow rate (from 0.5 to 2.5 μL min⁻¹). As illustrated in frames 2003 and 2004, a small flow rate (<0.5 μL min⁻¹) could result in a decreased signal upon successive measurement due to the molecule depletion, while at typical sweat rates (e.g., 1 μL min⁻¹) the sensor could provide stable and consistent readings.

### Example 6: Refreshing Time Analysis and Simulations

Concentration refreshing time T_{c} can be obtained by considering the mass balance of a standard well-mixed model: dC/dt+(C−Cᵢ)Q/Vᵣ=0, where Cᵢ and Q denote, respectively, the new solute concentration and total flow rate into the reservoir, and Vᵣ represents the reservoir volume. This simple ordinary differential equation can be solved analytically to obtain the solute concentration in the reservoir as a function of time t: C(t)=Cᵢ−(Cᵢ−C₀)e^{-Qt/V}^{ᵣ}, where C₀ is the initial concentration in the reservoir. Hence, the refreshing time taken for the reservoir to reach a concentration of kCᵢ can be readily calculated as

$${T}_{c} = \frac{{V}_{r}}{Q} ln \frac{1 - {C}_{0} / {C}_{i}}{1 - k} .$$

For an experimentally measured sweat rate of Q=1.5 μL min⁻¹ and concentration change from C₀=20 μM to Cᵢ=80 μM, we estimate that the designed reservoir volume Vᵣ=2 mm³ leads to a refreshing time T_{c}≈2.7 minutes to reach k=90% of the new concentration. This simple analysis provides an order of magnitude estimate of the required refreshing time by assuming perfect mixing. To obtain more realistic estimates, a three-dimensional model was created with the same geometry of the device. The mass transport process was simulated using a finite-element software COMSOL Multiphysics by numerically solving the Stokes equation for an incompressible flow:

∇p=μ∇²v, ∇·v=0,

coupled with the convection-diffusion equation

$$\frac{\partial C}{\partial t} + v \cdot ∇ C = D {∇}^{2} C .$$

Here p and v denote, respectively, pressure and flow velocity, whereas μ and D denote, respectively, solvent viscosity and solute diffusivity. The Stokes equation is applicable here because the Reynolds number is on the order of 10⁻² for this microfluidic device. The solute concentration in the chamber is tracked by computing the average concentration over the bottom surface of the chamber. A flow rate of 0.15 μL min⁻¹ is prescribed at each inlet, with the no-slip boundary condition on all channel walls. The simulated refreshing time as a function of number of inlets may be displayed. The refreshing time decreases as the number of inlets increases; for 10 inlets with a total inlet flow rate of 1.5 μL min⁻¹, the simulated 90% refreshing time T_{c}≈2.5 minutes, slightly less than the ballpark estimate based on perfect mixing (≈2.7 minutes). In FIG. 2 at frame 202, the concentration distribution over the bottom surface of the chamber is displayed at different time instances.

### Example 7: On-Body System Validation

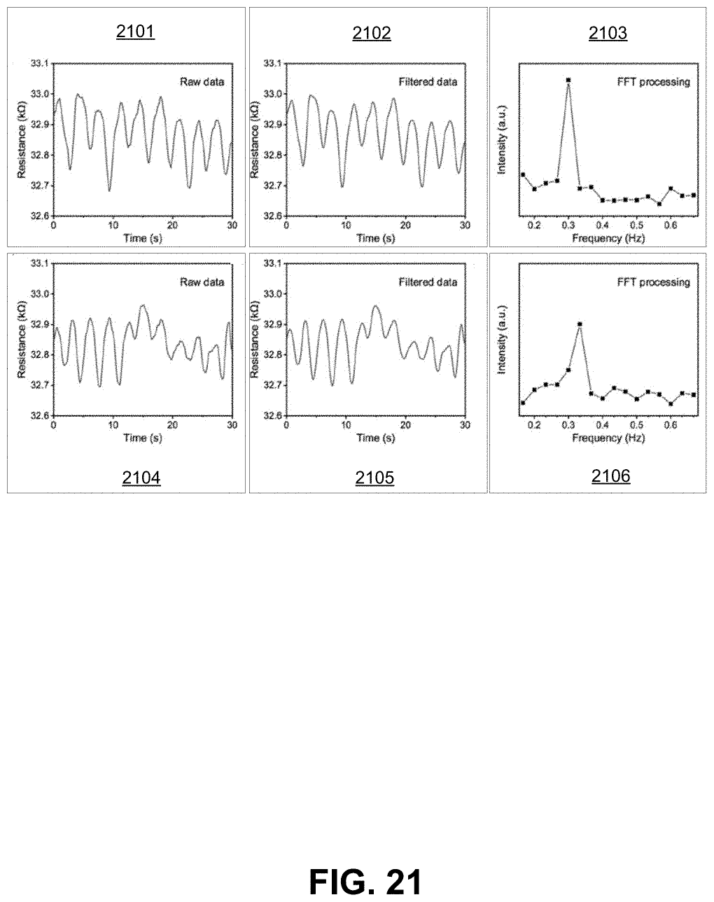

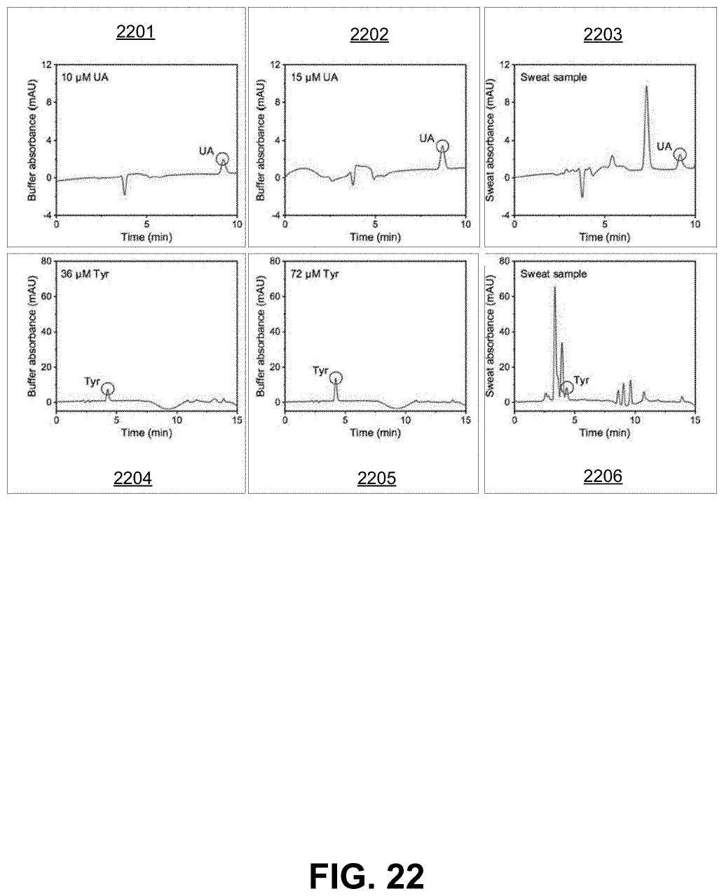

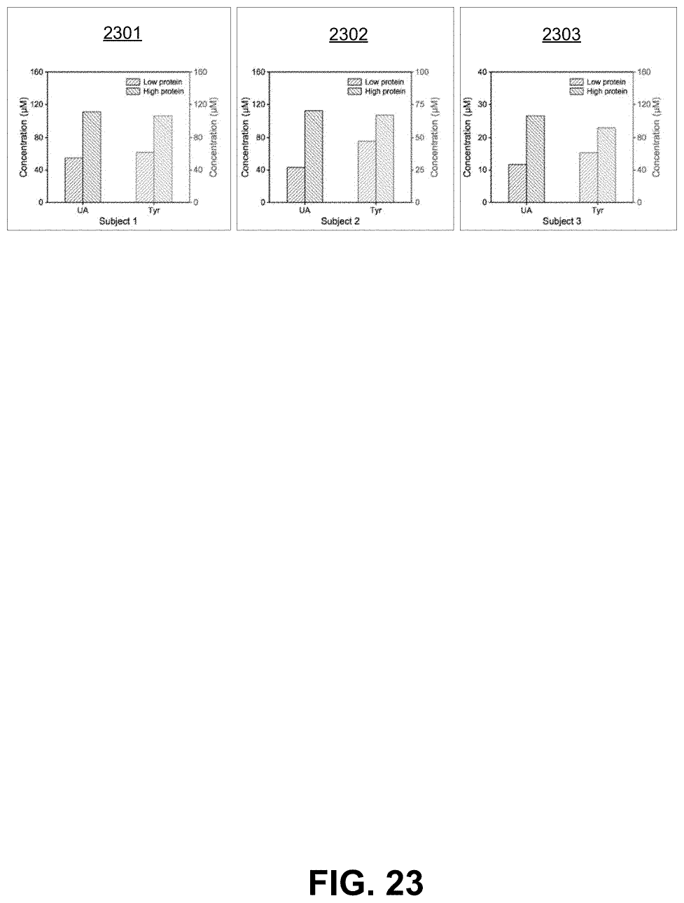

Constant-load cycling exercise was conducted on physically trained and untrained subjects (age range 18-40). The trained subjects (athletes) exercised regularly for at least 9 h per week while the untrained subjects had an average of 1 h of exercise per week. The subjects reported overnight fasting and were given a standardized protein drink. Two hours after protein consumption, the subjects' foreheads and necks were cleaned with alcohol swabs and gauze before the sensor patches were placed on-body. Stationary exercise bike was used for cycling trials. Subjects cycled at 60 rpm for 40 min. During the on-body trial, the data from the sensor patches were wirelessly sent to the user interface through Bluetooth. When the subjects started biking, the sensor system continuously acquired and transmitted physical sensor data at a rate of 10 Hz. Every minute, the electronic system initiated a transient voltage bias between the reference and working electrodes. If the bias triggered a current above an experimentally determined threshold, the system would start the 90-s DPV scan. The DPV scan was repeated every 5 minutes until the subject stopped biking. In between each DPV scan, physical sensor data was acquired. For the piezoresistive sensor, respiratory rate was calculated through spectral analysis by performing fast Fourier transform FFT on the respiration data. See FIG. 21, frames 2101-2106. The frequency corresponding to the largest amplitude in the frequency spectrum was converted to breaths per minute (bpm). Meanwhile sweat samples were collected periodically using centrifuge tubes for sensor validation via HPLC analysis. HPLC tests of UA and Tyr were done on HP Agilent 1100 HPLC using an Agilent Eclipse XDB-C18 5 μm 3×250 mm column. Tests of UA and Tyr were done with gradient methods. Detection wavelengths for UA and Tyr were 245 nm and 274 nm, respectively. Retention times were ˜9 min and ˜4 min for UA and Tyr, respectively. Sweat samples were diluted 3 times and serum samples were diluted 20 times, both with water. Results. Significantly lower sweat Tyr concentrations are observed from the trained athlete subjects (See FIG. 6 at frame 604). Validation of the chemical sensor analyzing UA and Tyr in raw sweat samples using HPLC analysis can be seen, for example, in FIG. 22, frames 2201-2206. Further system validations toward nutritional and metabolic monitoring are performed through a high/low protein diet study, higher levels of both sweat UA and Tyr are observed from all the subjects after a high protein diet See FIG. 23, frames 2301-2303.

### Example 8: Gout Management Study

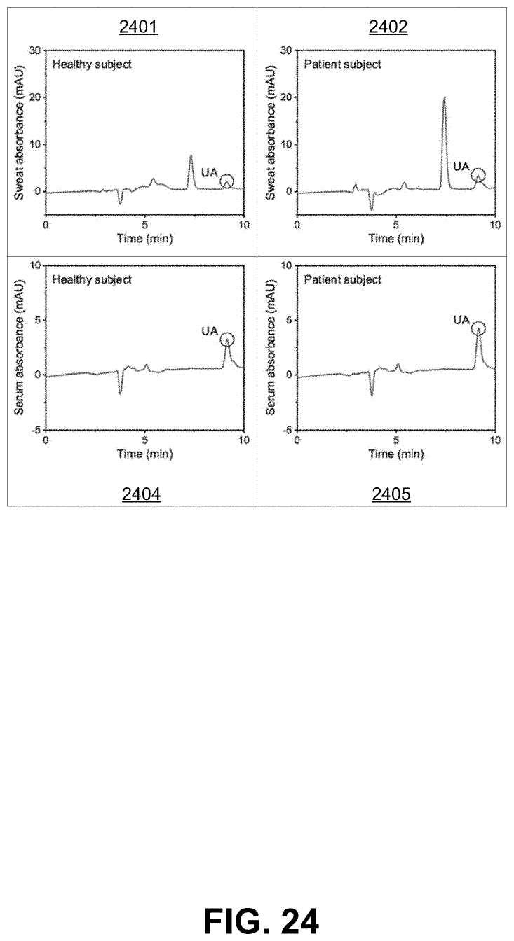

Methods. Purine-rich diet study was performed on both healthy male and female subjects (See FIG. 7, frames 701-704). The subjects reported to the lab after overnight fasting. Fresh blood drops were collected using a finger-prick approach. Constant load cycling exercise was conducted on the subjects with the sweat information collected by the multimodal sensing layer. The subjects were then given a purine-rich diet (250 g canned sardines) followed by a two-hour rest. The blood collection and the cycling trial were then repeated. The serum samples were obtained by standardized clotting of the blood sample for 90 min, and analyzed via HPLC. In order to further characterize the sweat UA sensing, two gout patients, two subjects with hyperuricemia, and two healthy subjects were recruited (See FIG. 7, at frame 705). The sweat samples and blood samples were collected 2 hours after their regular lunch and tested by the sensor patches and HPLC, respectively. For dynamic monitoring of UA before and after purine intake (see frame 706), a healthy subject underwent a blood collection and a cycling sweat test after overnight fasting, then consumed canned sardines. The blood collection and sweat test repeated periodically until 6 hours after the intake. The collected blood samples were analyzed with HPLC. The correlation plot in frame 707 was based on data obtained from 6 subjects (including 2 patients with gout). Results. Both the serum and sweat UA levels increase significantly after a purine-rich diet (frames 701-704) for the subjects with overnight fasting. Higher sweat and serum UA levels are observed from the male subject than the female subject. Further investigations indicate that significantly higher sweat UA levels are identified from the subjects with hyperuricemia and the patients with gouts compared to those from the healthy subjects, with a similar trend as their serum UA levels (frame 705 and also validated using HPLC as shown in FIG. 24, frames 2401-2405).

### Example 9: Circuitry Construction, Processing, and Wireless Transmission

Examples of circuitry that may be used in the systems and methods disclosed herein may be seen, for example, in FIG. 9. In various implementations, chemical sensing may include a microcontroller that generates the desired stimulation waveform (smoothened by an analog low pass filter (LPE)) to apply a specific sensing voltammogram on the electrodes through an external digital-to-analog converter (DAC). The resulting current response is amplified and converted to voltage by a transimpedance amplifier (TIA), and read by the built-in analog-to-digital converter (ADC). In implementations, responses of resistive sensors are acquired through voltage dividers and the ADC. The acquired data may then be wirelessly transmitted to the user over Bluetooth. An example method is described in more detail below. It is to be understood that this is only one implementation of the lab-on-skin biosensor disclosed according to various embodiments herein.

A 32-bit microcontroller with a built-in 12-bit analog-to-digital converter (ADC) may be used, for example, in accordance with various embodiments of the disclosure. For example, a microcontroller may be programmed with the ST-link/v2 in-circuit debugger and programmer. Through the Universal Asynchronous Receiver/Transmitter (UART) protocol, the microcontroller may receive commands and transmit acquired data to a Bluetooth transceiver module, which then may communicate with a user interface (mobile phone or laptop). For a DPV scan, for example, the microcontroller may control the DAC8552 digital-to-analog (DAC) converter through the Serial Peripheral Interface (SPI) protocol to output a steady reference potential (VR) for the reference electrode, and a dynamic working potential (VW) for the working electrode. A fourth order low pass filter (LPF) further may be used to stabilize the reference potential. The analog circuitry for the filter, potentiostat interface, and transimpedance amplifier (TIA) are shown for illustrative purposes in FIG. 9. The resulting current through the working electrode may be amplified and converted to voltage by the TIA, then read by the ADC peripheral of the microcontroller. The 12-bit ADC has a high sample rate of 5 million samples per second (Msps), allowing precise and time accurate measurements by taking averages. The microcontroller is also able to measure the resistance of the piezoresistive sensor, for example, through voltage divider circuits and the built-in ADC. The acquired multimodal data may be wirelessly transmitted via Bluetooth to the user device, and further analyzed via a custom developed software.

It is understood that the present invention is not limited to the specific details of these examples. While a preferred embodiment of the invention has been shown and described in considerable detail, it should be understood that many changes can be made in the structure without departing from the spirit or scope of the invention. Accordingly, it is not desired that the invention should be limited to the exact structure shown and described in the examples provided.

What is claimed is:

1. A lab-on-skin biosensor comprising:
a microfluidics layer;
a moisture resistant layer;
a multimodal sensing layer comprising an electrode; and
a logic circuit comprising a processor and a non-transitory memory with computer executable instructions embedded thereon;
wherein the microfluidics layer comprises multiple microchannels transversely oriented to channel a biological sample from a first surface of the microfluidics layer to a second surface of the microfluidics layer, the biological sample comprising a target molecule;
the moisture resistant layer couples to the first surface of the microfluidics layer and comprises an aperture to enable the biological sample to enter the microchannels of the microfluidics layer;
the multimodal sensing layer is fluidically coupled to the second surface of the microfluidics layer to receive the biological sample from the microchannels;
the electrode configured to detect a measurement of an electrical property corresponding to a target molecule being present in the biological sample; and
the logic circuit is electrically coupled to the electrode and the computer executable instructions cause the processor to identify the electrical property detected with the electrode when the target molecule is present in the biological sample.

2. The lab-on-skin biosensor of claim 1, wherein the multimodal sensing layer comprises polyimide film.

3. The lab-on-skin biosensor of claim 1, wherein the electrode comprises a catalytically active substrate.

4. The lab-on-skin biosensor of claim 3, wherein the catalytically active substrate is graphene.

5. The lab-on-skin biosensor of claim 1, wherein the electrical property is an electrical current.

6. The lab-on-skin biosensor of claim 1, wherein the electrical property is an electrical voltage.

7. The lab-on-skin biosensor of claim 1, wherein the electrical property is an electrical impedance.

8. The lab-on-skin biosensor of claim 1, wherein the computer executable instructions cause the processor to generate an indication identifying the presence of the target molecule based on the electrical property detected with the electrode.

9. The lab-on-skin biosensor of claim 8, further comprising a display, wherein the computer executable instructions further cause the processor to output the indication identifying the presence of the target molecule to the display.

10. The lab-on-skin biosensor of claim 10, wherein the moisture resistant layer comprises polyethylene terephthalate.

11. A method for manufacturing a lab-on-skin biosensor comprising a microfluidics layer, a moisture resistant layer, a multimodal sensing layer comprising an electrode, and a logic circuit, the method comprising:
shaping the microfluidics layer to channel a biological sample from a first surface of the microfluidics layer to a second surface of the microfluidics layer; and
laser scribing the electrode on a surface of a multimodal sensing layer;
the electrode configured to detect a measurement of an electrical property corresponding to a target molecule being present in the biological sample.

12. The method of claim 11, wherein shaping the microfluidics layer to channel a biological sample comprises laser engraving multiple microchannels.

13. The method of claim 11, further comprising electrodepositing a conductive substance onto a surface of the electrode.

14. The method of claim 11, further comprising engraving an aperture onto a surface of a moisture resistant layer.

15. The method of claim 11, wherein the multimodal sensing layer comprises polyimide film.

16. The method of claim 11, wherein the electrode comprises graphene.

17. A method for detecting a target molecule in a biological sample using a lab-on-skin biosensor comprised of a microfluidics layer comprising multiple microchannels transversely oriented to channel a biological sample from a first surface of the microfluidics layer to a second surface of the microfluidics layer, a moisture resistant layer, a multimodal sensing layer comprising an electrode and fluidically coupled to the microfluidics layer, and a logic circuit, the method comprising:
receiving, on a first surface of the microfluidics layer, a biological sample comprising the target molecule, such that the biological sample can be channeled from a first surface of the microfluidics layer to a second surface of the microfluidics layer;
obtaining, with the electrode, a measurement of an electrical property of the target molecule; and
generating, with the logic circuit, an indication that the target molecule is present in the biological sample based on the measurement of the electrical property.

18. The method of claim 18, further comprising sweeping the electrode to regenerate the multimodal sensing layer in-situ.

19. The method of claim 18, wherein the biological sample comprises one or more of sweat, tears, blood, urine, and saliva.

20. The method of claim 18, wherein the target molecule is a monoclonal antibody against an epitope of SARS-CoV-2.
